SEQUENCE LISTING (111 sequences)

SEQ ID NO: 1 - RNA, 22 nt, unidentified
  source 1..22: mol_type = mRNA; note = Artificial Sequence
tagcaccatctgaaatcggtta

SEQ ID NO: 2 - RNA, 23 nt, unidentified
  source 1..23: mol_type = mRNA; note = Artificial Sequence
tagcaccatttgaaatcagtgtt

SEQ ID NO: 3 - RNA, 22 nt, unidentified
  source 1..22: mol_type = mRNA; note = Artificial Sequence
tagcaccatttgaaatcggtta

SEQ ID NO: 4 - RNA, 22 nt, unidentified
  source 1..22: mol_type = mRNA; note = Artificial Sequence
taaccgatttcagatggtgcta

SEQ ID NO: 5 - RNA, 22 nt, unidentified
  source 1..22: mol_type = mRNA; note = Artificial Sequence
taaccgatttcagatggtgcta

SEQ ID NO: 6 - RNA, 22 nt, unidentified
  source 1..22: mol_type = mRNA; note = Artificial Sequence
taaccgatttcagatggtgcta

SEQ ID NO: 7 - RNA, 22 nt, unidentified
  source 1..22: mol_type = mRNA; note = Artificial Sequence
taaccgatttcagatggtgcta

SEQ ID NO: 8 - RNA, 21 nt, unidentified
  source 1..21: mol_type = mRNA; note = Artificial Sequence
taacgatttcagatggtgcta

SEQ ID NO: 9 - RNA, 22 nt, unidentified
  source 1..22: mol_type = mRNA; note = Artificial Sequence
taaccgatttcagatggtgcta

SEQ ID NO: 10 - RNA, 22 nt, unidentified
  source 1..22: mol_type = mRNA; note = Artificial Sequence
taaccgatttcagatggtgcta

SEQ ID NO: 11 - RNA, 22 nt, unidentified
  source 1..22: mol_type = mRNA; note = Artificial Sequence
taaccgatttcagatggtgcta

SEQ ID NO: 12 - RNA, 22 nt, unidentified
  source 1..22: mol_type = mRNA; note = Artificial Sequence
taaccgatttcagatggtgcta

SEQ ID NO: 13 - RNA, 22 nt, unidentified
  source 1..22: mol_type = mRNA; note = Artificial Sequence
taaccgatttcagatggtgcta

SEQ ID NO: 14 - RNA, 22 nt, unidentified
  source 1..22: mol_type = mRNA; note = Artificial Sequence
taaccgatttcagatggtgcta

SEQ ID NO: 15 - RNA, 22 nt, unidentified
  source 1..22: mol_type = mRNA; note = Artificial Sequence
taaccgatttcagatggtgcta

SEQ ID NO: 16 - RNA, 22 nt, unidentified
  source 1..22: mol_type = mRNA; note = Artificial Sequence
taaccgatttcagatggtgcta

SEQ ID NO: 17 - RNA, 22 nt, unidentified
  source 1..22: mol_type = mRNA; note = Artificial Sequence
taaccgatttcagatggtgcta

SEQ ID NO: 18 - RNA, 22 nt, unidentified
  source 1..22: mol_type = mRNA; note = Artificial Sequence
taaccgatttcagatggtgcta

SEQ ID NO: 19 - RNA, 22 nt, unidentified
  source 1..22: mol_type = mRNA; note = Artificial Sequence
taaccgatttcagatggtgcta

SEQ ID NO: 20 - RNA, 22 nt, unidentified
  source 1..22: mol_type = mRNA; note = Artificial Sequence
taaccgatttcagatggtgcta

SEQ ID NO: 21 - RNA, 22 nt, unidentified
  source 1..22: mol_type = mRNA; note = Artificial Sequence
taaccgatttcagatggtgcta

SEQ ID NO: 22 - RNA, 22 nt, unidentified
  source 1..22: mol_type = mRNA; note = Artificial Sequence
taaccgatttcagatggtgcta

SEQ ID NO: 23 - RNA, 22 nt, unidentified
  source 1..22: mol_type = mRNA; note = Artificial Sequence
taaccgatttcagatggtgcta

SEQ ID NO: 24 - RNA, 22 nt, unidentified
  source 1..22: mol_type = mRNA; note = Artificial Sequence
taaccgatttcagatggtgcta

SEQ ID NO: 25 - RNA, 22 nt, unidentified
  source 1..22: mol_type = mRNA; note = Artificial Sequence
taaccgatttcagatggtgcta

SEQ ID NO: 26 - RNA, 23 nt, unidentified
  source 1..23: mol_type = mRNA; note = Artificial Sequence
aacactgatttcaaatggtgcta

SEQ ID NO: 27 - RNA, 23 nt, unidentified
  source 1..23: mol_type = mRNA; note = Artificial Sequence
aacactgatttcaaatggtgcta

SEQ ID NO: 28 - RNA, 23 nt, unidentified
  source 1..23: mol_type = mRNA; note = Artificial Sequence
aacactgatttcaaatggtgcta

SEQ ID NO: 29 - RNA, 23 nt, unidentified
  source 1..23: mol_type = mRNA; note = Artificial Sequence
aacactgatttcaaatggtgcta

SEQ ID NO: 30 - RNA, 23 nt, unidentified
  source 1..23: mol_type = mRNA; note = Artificial Sequence
aacactgatttcaaatggtgcta

SEQ ID NO: 31 - RNA, 23 nt, unidentified
  source 1..23: mol_type = mRNA; note = Artificial Sequence
aacactgatttcaaatggtgcta

SEQ ID NO: 32 - RNA, 23 nt, unidentified
  source 1..23: mol_type = mRNA; note = Artificial Sequence
aacactgatttcaaatggtgcta

SEQ ID NO: 33 - RNA, 23 nt, unidentified
  source 1..23: mol_type = mRNA; note = Artificial Sequence
aacactgatttcaaatggtgcta

SEQ ID NO: 34 - RNA, 23 nt, unidentified
  source 1..23: mol_type = mRNA; note = Artificial Sequence
aacactgatttcaaatggtgcta

SEQ ID NO: 35 - RNA, 23 nt, unidentified
  source 1..23: mol_type = mRNA; note = Artificial Sequence
aacactgatttcaaatggtgcta

SEQ ID NO: 36 - RNA, 23 nt, unidentified
  source 1..23: mol_type = mRNA; note = Artificial Sequence
aacactgatttcaaatggtgcta

SEQ ID NO: 37 - RNA, 23 nt, unidentified
  source 1..23: mol_type = mRNA; note = Artificial Sequence
aacactgatttcaaatggtgcta

SEQ ID NO: 38 - RNA, 23 nt, unidentified
  source 1..23: mol_type = mRNA; note = Artificial Sequence
aacactgatttcaaatggtgcta

SEQ ID NO: 39 - RNA, 23 nt, unidentified
  source 1..23: mol_type = mRNA; note = Artificial Sequence
aacactgatttcaaatggtgcta

SEQ ID NO: 40 - RNA, 23 nt, unidentified
  source 1..23: mol_type = mRNA; note = Artificial Sequence
aacactgatttcaaatggtgcta

SEQ ID NO: 41 - RNA, 23 nt, unidentified
  source 1..23: mol_type = mRNA; note = Artificial Sequence
aacactgatttcaaatggtgcta

SEQ ID NO: 42 - RNA, 23 nt, unidentified
  source 1..23: mol_type = mRNA; note = Artificial Sequence
aacactgatttcaaatggtgcta

SEQ ID NO: 43 - RNA, 23 nt, unidentified
  source 1..23: mol_type = mRNA; note = Artificial Sequence
aacactgatttcaaatggtgcta

SEQ ID NO: 44 - RNA, 23 nt, unidentified
  source 1..23: mol_type = mRNA; note = Artificial Sequence
aacactgatttcaaatggtgcta

SEQ ID NO: 45 - RNA, 23 nt, unidentified
  source 1..23: mol_type = mRNA; note = Artificial Sequence
aacactgatttcaaatggtgcta

SEQ ID NO: 46 - RNA, 23 nt, unidentified
  source 1..23: mol_type = mRNA; note = Artificial Sequence
aacactgatttcaaatggtgcta

SEQ ID NO: 47 - RNA, 23 nt, unidentified
  source 1..23: mol_type = mRNA; note = Artificial Sequence
aacactgatttcaaatggtgcta

SEQ ID NO: 48 - RNA, 23 nt, unidentified
  source 1..23: mol_type = mRNA; note = Artificial Sequence
aacactgatttcaaatggtgcta

SEQ ID NO: 49 - RNA, 23 nt, unidentified
  source 1..23: mol_type = mRNA; note = Artificial Sequence
aacactgatttcaaatggtgcta

SEQ ID NO: 50 - RNA, 22 nt, unidentified
  source 1..22: mol_type = mRNA; note = Artificial Sequence
taaccgatttcaaatggtgcta

SEQ ID NO: 51 - RNA, 22 nt, unidentified
  source 1..22: mol_type = mRNA; note = Artificial Sequence
taaccgatttcaaatggtgcta

SEQ ID NO: 52 - RNA, 22 nt, unidentified
  source 1..22: mol_type = mRNA; note = Artificial Sequence
taaccgatttcaaatggtgcta

SEQ ID NO: 53 - RNA, 22 nt, unidentified
  source 1..22: mol_type = mRNA; note = Artificial Sequence
taaccgatttcaaatggtgcta

SEQ ID NO: 54 - RNA, 22 nt, unidentified
  source 1..22: mol_type = mRNA; note = Artificial Sequence
taaccgatttcaaatggtgcta

SEQ ID NO: 55 - RNA, 22 nt, unidentified
  source 1..22: mol_type = mRNA; note = Artificial Sequence
taaccgatttcaaatggtgcta

SEQ ID NO: 56 - RNA, 22 nt, unidentified
  source 1..22: mol_type = mRNA; note = Artificial Sequence
taaccgatttcaaatggtgcta

SEQ ID NO: 57 - RNA, 22 nt, unidentified
  source 1..22: mol_type = mRNA; note = Artificial Sequence
taaccgatttcaaatggtgcta

SEQ ID NO: 58 - RNA, 22 nt, unidentified
  source 1..22: mol_type = mRNA; note = Artificial Sequence
taaccgatttcaaatggtgcta

SEQ ID NO: 59 - RNA, 22 nt, unidentified
  source 1..22: mol_type = mRNA; note = Artificial Sequence
taaccgatttcaaatggtgcta

SEQ ID NO: 60 - RNA, 22 nt, unidentified
  source 1..22: mol_type = mRNA; note = Artificial Sequence
taaccgatttcaaatggtgcta

SEQ ID NO: 61 - RNA, 22 nt, unidentified
  source 1..22: mol_type = mRNA; note = Artificial Sequence
taaccgatttcaaatggtgcta

SEQ ID NO: 62 - RNA, 22 nt, unidentified
  source 1..22: mol_type = mRNA; note = Artificial Sequence
taaccgatttcaaatggtgcta

SEQ ID NO: 63 - RNA, 22 nt, unidentified
  source 1..22: mol_type = mRNA; note = Artificial Sequence
taaccgatttcaaatggtgcta

SEQ ID NO: 64 - RNA, 22 nt, unidentified
  source 1..22: mol_type = mRNA; note = Artificial Sequence
taaccgatttcaaatggtgcta

SEQ ID NO: 65 - RNA, 22 nt, unidentified
  source 1..22: mol_type = mRNA; note = Artificial Sequence
taaccgatttcaaatggtgcta

SEQ ID NO: 66 - RNA, 22 nt, unidentified
  source 1..22: mol_type = mRNA; note = Artificial Sequence
taaccgatttcaaatggtgcta

SEQ ID NO: 67 - RNA, 22 nt, unidentified
  source 1..22: mol_type = mRNA; note = Artificial Sequence
taaccgatttcaaatggtgcta

SEQ ID NO: 68 - RNA, 22 nt, unidentified
  source 1..22: mol_type = mRNA; note = Artificial Sequence
taaccgatttcaaatggtgcta

SEQ ID NO: 69 - RNA, 22 nt, unidentified
  source 1..22: mol_type = mRNA; note = Artificial Sequence
taaccgatttcaaatggtgcta

SEQ ID NO: 70 - RNA, 22 nt, unidentified
  source 1..22: mol_type = mRNA; note = Artificial Sequence
taaccgatttcaaatggtgcta

SEQ ID NO: 71 - RNA, 22 nt, unidentified
  source 1..22: mol_type = mRNA; note = Artificial Sequence
taaccgatttcaaatggtgcta

SEQ ID NO: 72 - RNA, 22 nt, unidentified
  source 1..22: mol_type = mRNA; note = Artificial Sequence
taaccgatttcaaatggtgcta

SEQ ID NO: 73 - RNA, 22 nt, unidentified
  source 1..22: mol_type = mRNA; note = Artificial Sequence
taaccgatttcaaatggtgcta

SEQ ID NO: 74 - RNA, 23 nt, unidentified
  source 1..23: mol_type = mRNA; note = Artificial Sequence
aacaccgatttcaaatggtgcta

SEQ ID NO: 75 - RNA, 23 nt, unidentified
  source 1..23: mol_type = mRNA; note = Artificial Sequence
aacaccgatttcaaatggtgcta

SEQ ID NO: 76 - RNA, 23 nt, unidentified
  source 1..23: mol_type = mRNA; note = Artificial Sequence
aacaccgatttcaaatggtgcta

SEQ ID NO: 77 - RNA, 23 nt, unidentified
  source 1..23: mol_type = mRNA; note = Artificial Sequence
aacaccgatttcaaatggtgcta

SEQ ID NO: 78 - RNA, 23 nt, unidentified
  source 1..23: mol_type = mRNA; note = Artificial Sequence
aacaccgatttcaaatggtgcta

SEQ ID NO: 79 - RNA, 23 nt, unidentified
  source 1..23: mol_type = mRNA; note = Artificial Sequence
aacaccgatttcaaatggtgcta

SEQ ID NO: 80 - RNA, 23 nt, unidentified
  source 1..23: mol_type = mRNA; note = Artificial Sequence
aacaccgatttcaaatggtgcta

SEQ ID NO: 81 - RNA, 23 nt, unidentified
  source 1..23: mol_type = mRNA; note = Artificial Sequence
aacaccgatttcaaatggtgcta

SEQ ID NO: 82 - RNA, 23 nt, unidentified
  source 1..23: mol_type = mRNA; note = Artificial Sequence
aacaccgatttcaaatggtgcta

SEQ ID NO: 83 - RNA, 23 nt, unidentified
  source 1..23: mol_type = mRNA; note = Artificial Sequence
aacaccgatttcaaatggtgcta

SEQ ID NO: 84 - RNA, 23 nt, unidentified
  source 1..23: mol_type = mRNA; note = Artificial Sequence
aacaccgatttcaaatggtgcta

SEQ ID NO: 85 - RNA, 23 nt, unidentified
  source 1..23: mol_type = mRNA; note = Artificial Sequence
aacaccgatttcaaatggtgcta

SEQ ID NO: 86 - RNA, 23 nt, unidentified
  source 1..23: mol_type = mRNA; note = Artificial Sequence
aacaccgatttcaaatggtgcta

SEQ ID NO: 87 - RNA, 23 nt, unidentified
  source 1..23: mol_type = mRNA; note = Artificial Sequence
aacaccgatttcaaatggtgcta

SEQ ID NO: 88 - RNA, 23 nt, unidentified
  source 1..23: mol_type = mRNA; note = Artificial Sequence
aacaccgatttcaaatggtgcta

SEQ ID NO: 89 - RNA, 23 nt, unidentified
  source 1..23: mol_type = mRNA; note = Artificial Sequence
aacaccgatttcaaatggtgcta

SEQ ID NO: 90 - RNA, 23 nt, unidentified
  source 1..23: mol_type = mRNA; note = Artificial Sequence
aacaccgatttcaaatggtgcta

SEQ ID NO: 91 - RNA, 23 nt, unidentified
  source 1..23: mol_type = mRNA; note = Artificial Sequence
aacaccgatttcaaatggtgcta

SEQ ID NO: 92 - RNA, 23 nt, unidentified
  source 1..23: mol_type = mRNA; note = Artificial Sequence
aacaccgatttcaaatggtgcta

SEQ ID NO: 93 - RNA, 23 nt, unidentified
  source 1..23: mol_type = mRNA; note = Artificial Sequence
aacaccgatttcaaatggtgcta

SEQ ID NO: 94 - RNA, 23 nt, unidentified
  source 1..23: mol_type = mRNA; note = Artificial Sequence
aacaccgatttcaaatggtgcta

SEQ ID NO: 95 - RNA, 23 nt, unidentified
  source 1..23: mol_type = mRNA; note = Artificial Sequence
aacaccgatttcaaatggtgcta

SEQ ID NO: 96 - RNA, 23 nt, unidentified
  source 1..23: mol_type = mRNA; note = Artificial Sequence
aacaccgatttcaaatggtgcta

SEQ ID NO: 97 - RNA, 23 nt, unidentified
  source 1..23: mol_type = mRNA; note = Artificial Sequence
aacaccgatttcaaatggtgcta

SEQ ID NO: 98 - RNA, 23 nt, unidentified
  source 1..23: mol_type = mRNA; note = Artificial Sequence
aacaccgatttcaaatggtgcta

SEQ ID NO: 99 - RNA, 23 nt, unidentified
  source 1..23: mol_type = mRNA; note = Artificial Sequence
aacaccgatttcaaatggtgcta

SEQ ID NO: 100 - RNA, 23 nt, unidentified
  source 1..23: mol_type = mRNA; note = Artificial Sequence
aacaccgatttcaaatggtgcta

SEQ ID NO: 101 - RNA, 23 nt, unidentified
  source 1..23: mol_type = mRNA; note = Artificial Sequence
aacaccgatttcaaatggtgcta

SEQ ID NO: 102 - RNA, 23 nt, unidentified
  source 1..23: mol_type = mRNA; note = Artificial Sequence
aacaccgatttcaaatggtgcta

SEQ ID NO: 103 - RNA, 23 nt, unidentified
  source 1..23: mol_type = mRNA; note = Artificial Sequence
aacaccgatttcaaatggtgcta

SEQ ID NO: 104 - RNA, 23 nt, unidentified
  source 1..23: mol_type = mRNA; note = Artificial Sequence
aacaccgatttcaaatggtgcta

SEQ ID NO: 105 - RNA, 23 nt, unidentified
  source 1..23: mol_type = mRNA; note = Artificial Sequence
aacaccgatttcaaatggtgcta

SEQ ID NO: 106 - RNA, 23 nt, unidentified
  source 1..23: mol_type = mRNA; note = Artificial Sequence
aacaccgatttcaaatggtgcta

SEQ ID NO: 107 - RNA, 23 nt, unidentified
  source 1..23: mol_type = mRNA; note = Artificial Sequence
aacaccgatttcaaatggtgcta

SEQ ID NO: 108 - protein, 26 aa, unidentified
  source 1..26: note = Artificial Sequence
KKALLALALHHLAHLALHLALALKKA

SEQ ID NO: 109 - protein, 12 aa, unidentified
  source 1..12: note = Artificial Sequence
GRKKRRQRRRCG

SEQ ID NO: 110 - protein, 27 aa, unidentified
  source 1..27: note = Artificial Sequence
GALFLGFLGAAGSTMGAWSQPKKKRKV

SEQ ID NO: 111 - protein, 11 aa, unidentified
  source 1..11: note = Artificial Sequence
ACTGSTQHQCG

(19) United States
(12) Patent Application Publication
(10) Pub. No.: US 2025/0304956 A1
(43) Pub. Date: Oct. 2, 2025

(54) miRNA-BASED COMPOSITIONS AND METHODS OF USE THEREOF

(71) Applicant: LeaderMed Champion Limited, Wan Chai (CN)

(72) Inventors: Jie Jenny Yuan, Branchburg, NJ (US); Qiao Joanne Jiang Chen, Pennington, NJ (US); Peiwen Sun, St. Louis, MO (US)

(73) Assignee: LeaderMed Champion Limited, Wan Chai (CN)

(21) Appl. No.: 18/681,240

(22) PCT Filed: Aug. 4, 2022
(86) PCT No.: PCT/IB2022/057268

§ 371 Date: Feb. 5, 2024

Related U.S. Application Data
(60) Provisional application No. 63/230,502, filed on Aug. 6, 2021.

(51) Int. Cl. C12N 15/113; A61K 9/00; A61K 9/1272; A61K 31/713; A61P 17/00

(52) U.S. Cl. CPC C12N 15/113; A61K 9/0014; A61K 9/1272; A61K 31/713; A61P 17/00; C12N 2310/113; C12N 2310/14; C12N 2310/531

(57) ABSTRACT

This disclosure relates to agents and methods for treating a condition or disorder associated with collagen deficiency, for preventing or treating a skin disease or disorder or for improving a skin condition.

### CROSS-REFERENCE TO RELATED APPLICATIONS

This application is the U.S. national phase of International Patent Application No. PCT/IB2022/057268, filed Aug. 4, 2022, which claims priority under 35 U.S.C. § 119(e) to U.S. Provisional Patent Application No. 63/230,502, filed Aug. 6, 2021. The foregoing applications are incorporated by reference herein in their entirety.

### REFERENCE TO AN ELECTRONIC SEQUENCE LISTING

The contents of the electronic sequence listing (346882.00102SeqList2.xml; Size: 117,881 bytes; and Date of Creation: Jan. 28, 2025) is herein incorporated by reference in its entirety.

### FIELD OF THE INVENTION

This invention relates generally to agents and methods for treating a condition or disorder associated with collagen deficiency, for preventing or treating a skin disease or disorder, or for improving a skin condition.

### BACKGROUND OF THE INVENTION

Topical use of therapeutic nucleic acids, such as plasmid DNA and small interfering RNA (siRNA), has been increasingly studied due to the importance of treating undesired skin aging appearance, physiology or structural changes. For example, siRNA is investigated as a novel drug for allergic skin diseases due to its target-factor silencing effect. However, topical application of naked siRNA does not exert strong therapeutic effects due to its low delivery efficiency to target tissues and cells by various skin barriers like stratum corneum and epidermis, and due to its degradation by enzymes in the body. Therefore, there is a strong need for nucleic acid therapeutics and transdermal delivery systems to facilitate the nucleic acid therapeutics passing through skin barriers, protect them from degradation, and deliver them into target cells.

### SUMMARY OF THE INVENTION

This disclosure addresses the need mentioned above in a number of aspects.

In one aspect, this disclosure provides a composition comprising an agent for treating a condition or disorder associated with collagen deficiency, or for preventing or treating a skin disease or disorder, or for improving a skin condition. In some embodiments, the agent comprises an antagonist of at least one of miR-29a, miR-29b, and miR-29c. In some embodiments, the antagonist is capable of increasing collagen production in a skin cell by reducing a level or activity of at least one of the miR-29a, the miR-29b, and the miR-29c. In some embodiments, the composition comprises: a liposome formulation comprising a phospholipid, a cationic lipid, a pH-dependent cationic lipid, or a combination thereof. In some embodiments, the composition comprises a niosome formulation comprising a hydrated nonionic surfactant. In some embodiments, the composition comprises a polymer formulation comprising a positively charged polymer.

In some embodiments, the antagonist comprises an antagomir of the miR-29a, the miR-29b, or the miR-29c, an antisense oligonucleotide targeting a mature sequence of the miR-29a, the miR-29b, or the miR-29c, an inhibitory RNA molecule, or a combination thereof. In some embodiments, the miR-29a comprises the polynucleotide sequence of SEQ ID NO: 1. In some embodiments, the miR-29b comprises the polynucleotide sequence of SEQ ID NO: 2. In some embodiments, the miR-29c comprises the polynucleotide sequence of SEQ ID NO: 3. In some embodiments, the antagonist comprises a polynucleotide sequence of SEQ ID NOs: 4-107. In some embodiments, the inhibitory RNA molecule comprises a siRNA or a shRNA that comprises the mature sequence of the miR-29a, the miR-29b, or the miR-29c. In some embodiments, two or more of the antisense oligonucleotides targeting a mature sequence of the miR-29a, the antisense oligonucleotide targeting a mature sequence of the miR-29b, and the antisense oligonucleotide targeting a mature sequence of the miR-29b are carried on the same nucleic acid molecule.

In some embodiments, the liposome formulation comprises phospholipid, cholesterol, PEG or a derivative thereof, or a combination thereof. In some embodiments, the liposome formulation comprises phospholipid, cholesterol, and PEG or a derivative thereof. In some embodiments, the phospholipid has a chain of 16 to 22 carbons. In some embodiments, the phospholipid comprises hydrogenated soy phosphatidylcholine (HSPC), distearoylphosphatidylcholine (DSPC), 1,2-dioleoyl-sn-glycero-3-phosphocholine (DOPC), 1,2-distearoyl-3-sn-glycerophosphoethanolamine (DSPE), or dioleoylphosphatidylethanolamine (DOPE). In some embodiments, the PEG has a molecular weight of 120 Daltons to 5000 Daltons. In some embodiments, the PEG comprises PEG[N-(carbamoyl-methoxypolyethylene glycol XXX)-1, 2-distearoyl-sn-glycero-3-phosphoethanolamine sodium salt]. In some embodiments, the liposome formulation comprises 40-90 wt % of phospholipid, 10-60 wt % of cholesterol, and 0-7 wt % PEG. In some embodiments, the liposome formulation, the noisome formulation, or the polymer formulation comprises a cell-penetrating peptide. In some embodiments, the cell-penetrating peptide comprises an amino acid sequence of SEQ ID NOs: 108.

In some embodiments, the liposome formulation comprises: (i) 0-55 wt % of cationic lipid; (ii) 40-90 wt % of the cationic lipid and 10-60 wt % of cholesterol; or (iii) 0-8 wt % of PEG. In some embodiments, the cationic lipid comprises N-[1-(2,3-dioleoloxy)propyl]-N,N,N-trimethyl ammonium chloride (DOTAP), dimethyldioctadecylammonium (bromide salt) (DDAB), or a combination thereof.

In some embodiments, the liposome formulation comprises 0-55 wt % of pH-dependent cationic lipid. In some embodiments, the pH-dependent cationic lipid comprises 1,2-dioleoyl-3-dimethylammonium-propane (DODAP), N-palmitoyl homocysteine (PHC), or a combination thereof. In some embodiments, the liposome formulation comprises an edge activator or inorganic particle. In some embodiments, the edge activator or inorganic particle comprises sodium cholate, Span, Tween, and carbonate apatite. In some embodiments, the liposome formulation comprises a skin penetration enhancer. In some embodiments, the liposome formulation comprises 20-45 wt % of the skin penetration enhancer. In some embodiments, the skin penetration enhancer comprises ethanol.

In some embodiments, the nonionic surfactant comprises Span, Tween, brijs, alkyl amides, sorbitan ester, crown ester, or polyoxyethylene alkyl ether.

In some embodiments, the positively charged polymer comprises: (a) diethylaminoethylen (DEAE)—Dextran (DEAE-Dextran); (b) linear and branched polyethylenimine (PEI) or derivative thereof; (c) poly(dl-lactide-co-glycolide) (PLGA); (d) Chitosan and modified Chitoson; (e) β-Cyclodextrin; (f) polypeptides; (g) poly{N-[N-(2-aminoethyl)-2-aminoethyl]aspartamide}[PAsp(DET)]; (h) polylysine partially substituted with histidyl residues; and/or (i) linear cationic amphipathic histidine-rich peptide or derivative thereof; and/or a dendrimer. In some embodiments, the linear cationic amphipathic histidine-rich peptide comprises the amino acid sequence of SEQ ID NO: 109 or 110. In some embodiments, the dendrimer comprises poly(amidoamine) (PAMAM), poly(propylenimine) (PPI), or a derivative thereof.

In some embodiments, the composition further comprises a positively charged polycation. In some embodiments, the composition further comprises a targeting ligand. In some embodiments, the target ligand comprises (a) a fibroblast growth factor or fibronectin; or (b) a synthetic analog of luteinizing hormone-releasing hormone targeting peptide. In some embodiments, the composition further comprises a second agent. In some embodiments, the second agent comprises an anti-inflammatory agent or an antibiotic. In some embodiments, the composition is formulated as a gel, cream, lotion, or ointment.

In another aspect, this disclosure provides a kit or device comprising the composition as described herein. In some embodiments, the kit or device comprises a medical device, such as an implantable medical device. In some embodiments, the kit or device comprises a suture, a wound management patch, an injectable material, an implant device, a wound closure strip, or a surgical glue.

In another aspect, this disclosure also provides a method for treating a condition or disorder associated with collagen deficiency, for preventing or treating a skin disease or disorder, or for improving a skin condition in a subject. In some embodiments, the method comprises administering to the subject an effective amount of a composition comprising an antagonist of at least one of miR-29a, miR-29b, and miR-29c. In some embodiments, the antagonist is capable of increasing collagen production in a skin cell by reducing a level or activity of at least one of the miR-29a, the miR-29b, and the miR-29c.

In yet another aspect, this disclosure further provides a method of increasing collagen production in a skin cell of a subject. In some embodiments, the method comprises administering to the subject an effective amount of a composition comprising an antagonist of at least one of the miR-29a, the miR-29b, and the miR-29c. In some embodiments, the antagonist is capable of reducing a level or activity of at least one of the miR-29a, the miR-29b, and the miR-29c.

In some embodiments, the method further comprises administering to the subject a second agent. In some embodiments, the second agent comprises an anti-inflammatory agent or an antibiotic. In some embodiments, the second agent is administered to the subject before, after, or concomitantly with application of the composition.

In some embodiments, the skin condition is selected from skin aging, alopecia, scar, acne, actinic damage, dandruff, eczema, fine lines, psoriasis, warts, and wrinkles.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows the results of quantitative analysis of collagen I immunofluorescence. NT: non-transfected fibroblasts culture. VC: fibroblasts culture transfected with empty vector+ vitamin C. Mock: fibroblasts culture transfected with empty vector. Sample 1 is fibroblasts culture transfected with miR-29 vector. Sample 2 is fibroblasts culture transfected with anti-miR-29. Immunofluorescence scores are represented as positive ratios (positive area/total cell count).

### DETAILED DESCRIPTION OF THE INVENTION

This disclosure provides agents (e.g., nucleic acid therapeutics) and compositions thereof capable of modulating expression or function of specific genes and thus treating a condition or disorder associated with collagen deficiency or improving skin conditions or treating skin diseases, such as wrinkle, alopecia, and scar. The compositions may include delivery systems for delivery (e.g., transdermal delivery) of the agents into target cells of skin tissues to modulate expression or function of specific genes, resulting in improvement of skin conditions or treatment of skin diseases. The condition or disorder associated with collagen deficiency may be a condition or disorder of a skin, hair, nail, bone, or joint of a subject.

### Agents and Compositions for Transdermal Delivery

a. Agents

In one aspect, this disclosure provides agents and compositions thereof capable of modulating expression or function of specific genes and thus improving skin conditions.

miRNAs have been identified as key players in the molecular pathogenesis of skin diseases, and in the manifestation of various skin conditions such as aging, pigmentation disorders, acne, and skin senescence. In some embodiments, the agents may include miRNA mimics (miRNA replacement therapy) and miRNA inhibitors (antagomiR therapy) that are capable of improving skin conditions or treating skin diseases or disorders, e.g., regulating or treating pigmentation, skin aging, UV damage to skin, acne, psoriasis, and apotic dermatitis.

In some embodiments, the agents may include DNA, DNAzyme, oligonucleotides, mRNA, microRNA, siRNA or a combination thereof. In some embodiments, the agents may include nucleic acids that are single-targeted or multi-targeted, such as a physical “cocktail” or chemical linkage thereof. In some embodiments, the agents may include enzymes, proteins, receptors, transcription factors, mRNAs, or a combination thereof. In some embodiments, the agents are capable of modulating (e.g., increasing or decreasing) expression or function of specific genes that have a role in skin aging, skin repair, or skin diseases.

This disclosure is based, at least in part, on the unexpected discovery that the microRNA 29 (or miR-29) family (e.g., miR-29a-c), which is down-regulated in the heart in response to stress, regulates collagen deposition. Up-regulation of miR-29a-c (including miR-29a, miR-29b, and miR-29c) expression or function results in decrease of expression of collagen and fibrin genes. On the other hand, down-regulation of miR-29a-c expression or function leads to an increase in collagen generation or deposition.

miR-29 is a family of microRNAs that consists of four known members, miR-29a, miR-29b1, miR-29b2, and miR-29c. miR-29b1 and miR-29b2 are identical. While miR-29b-1 and miR-29a stem from the same transcript originating from chromosome 7 in humans and chromosome 6 in mice, the miRNA cluster containing miR-29b-2 and miR-29c is transcribed from chromosome 1 in both species. The mature miRNA sequences for each of the human miR-29 family members is listed in Table 1.

Target determination for the miR-29 family revealed that the miR-29 family shows a high preference for targeting genes involved in collagen formation as well as other extracellular matrix proteins, such as collagen type I, C1 and C2 (COL1A1, COL1A2) collagen type III, C1 (COL3A1), elastin (ELN), fibrillin 1 (FBN1), metallopeptidases, and integrins. Thus, the miR-29 family is likely to play an active role in skin remodeling process, including modulation of collagen production and/or deposition.

Accordingly, one aspect of the disclosure is antagonism of miR-29a-c expression or activity. Antagonism may involve introducing exogenous miR-29a-c inhibitors into the skin fibroblasts or other tissues of interest, either directly using naked nucleic acid, through gene expression or a transdermal delivery system.

This disclosure further provides compositions and methods of use thereof for stimulating collagen production in skin cells in a subject in need thereof. The compositions comprise one or more antagonists of miR-29a-c that are capable of down-regulating expression or function of miR-29a-c. Additionally, this disclosure also provides a method of inducing collagen deposition in tissue. The method may include contacting the tissue with an antagonist of miR-29a-c.

In some embodiments, the composition may include an antagonist of at least one of miR-29a, miR-29b, and miR-29c. In some embodiments, the antagonist is capable of increasing collagen production in a skin cell (or collagen deposition in a tissue) by reducing a level or activity of at least one of the miR-29a, the miR-29b, and the miR-29c. In some embodiments, the miR-29a may include the polynucleotide sequence of SEQ ID NO: 1 or a polynucleotide sequence having at least 80% (e.g., 80%, 85%, 90%, 95%, 99%) sequence identity with SEQ ID NO: 1. In some embodiments, the miR-29b may include the polynucleotide sequence of SEQ ID NO: 2 or a polynucleotide sequence having at least 80% (e.g., 80%, 85%, 90%, 95%, 99%) sequence identity with SEQ ID NO: 2. In some embodiments, the miR-29c may include the polynucleotide sequence of SEQ ID NO: 3 or a polynucleotide sequence having at least 80% (e.g., 80%, 85%, 90%, 95%, 99%) sequence identity with SEQ ID NO: 3.

TABLE 1
Example miR-29a-c Sequences

| SEQ | | |
|---|---|---|
| ID | | |
| NO | Sequence | miR-29 Target |
| 1 | UAGCACCAUCUGAAAUCGGUUA | hsa-miR-29a |
| 2 | UAGCACCAUUUGAAAUCAGUGUU | hsa-miR-29b-1 & b-2 |
| 3 | UAGCACCAUUUGAAAUCGGUUA | hsa-miR-29c |

In some embodiments, non-limiting examples of the antagonist may include an antagomir of the miR-29a, the miR-29b, or the miR-29c, an antisense oligonucleotide targeting a mature sequence of the miR-29a, the miR-29b, or the miR-29c, an inhibitory RNA molecule, or a combination thereof.

In some embodiments, the function of miRNAs may be inhibited by the administration of antagomirs. Antagomirs may be single-stranded, chemically modified ribonucleotides that are at least partially complementary to a miRNA sequence (e.g., miR-29a-c). Antagomirs may comprise one or more modified nucleotides, such as 2′-O-methyl-Sugar modifications. In some embodiments, antagomirs comprise only modified nucleotides. Antagomirs may also comprise one or more phosphorothioate linkages resulting in a partial or full phosphorothioate backbone. To facilitate in vivo delivery and stability, the antagomir may be linked to a cholesterol moiety at its 3′ end. Antagomirs suitable for inhibiting miRNAs may be about 15 to about 50 nucleotides in length (e.g., about 18 to about 30 nucleotides in length, about 20 to about 25 nucleotides in length). “Partially complementary” refers to a sequence that is at least about 75%, 80%, 85%, 90%, 95%, 96%, 97%, 98%, or 99% complementary to a target polynucleotide sequence. The antagomirs may be at least about 75%, 80%, 85%, 90%, 95%, 96%, 97%, 98%, or 99% complementary to a mature miRNA sequence. In some embodiments, the antagomir may be substantially complementary to a mature miRNA sequence, that is at least about 95%, 96%, 97%, 98%, or 99% complementary to a target polynucleotide sequence. In other embodiments, the antagomirs are 100% complementary to the mature miRNA sequence.

In some embodiments, the antagonist of miR-29a-c may be an antagomir. The antagomir may comprise a sequence that is at least partially complementary to a mature miRNA sequence of miR-29a, miR-29b, or miR-29c. In some embodiments, the antagomir comprises a sequence that is at least partially complementary to the sequence of SEQ ID NO: 1, SEQ ID NO: 2, or SEQ ID NO: 3. In some embodiments, the antagomir comprises a sequence that is at 80% to 100% (e.g., 80%, 85%, 90%, 95%, 96%, 97%, 98%, 99%, 100%) complementary to SEQ ID NO: 1, SEQ ID NO: 2, or SEQ ID NO: 3.

In some embodiments, the antagonist of miR-29a-c may be an antisense oligonucleotide targeting a mature sequence of miR-29a, miR-29b or miR-29c. The antisense oligonucleotides may be ribonucleotides or deoxyribonucleotides. In some embodiments, the antisense oligonucleotides may have at least one chemical modification. In some embodiments, antisense oligo nucleotides may include one or more “locked nucleic acids (LNAs).” LNAs are modified ribonucleotides that contain an extra bridge between the 2′ and 4′ carbons of the ribose sugar moiety resulting in a “locked’ conformation that confers enhanced thermal stability to oligonucleotides containing the LNAs. Alternatively, the antisense oligonucleotides may include peptide nucleic acids (PNAs), which contain a peptide-based backbone rather than a sugar-phosphate backbone. Other chemical modifications that the antisense oligonucleotides may contain include, but are not limited to, sugar modifications, such as 2′-O-alkyl (e.g., 2′-O-methyl. 2′-O-methoxyethyl), 2′-fluoro, and 4′ thiomodifications, and backbone modifications, such as one or more phosphorothioate, morpholino, or phosphonocarboxy late linkages (see, for example, U.S. Pat. Nos. 6,693,187 and 7,067,641, which are herein incorporated by reference in their entireties). In some embodiments, suitable antisense oligo nucleotides are 2′-O-methoxyethyl “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, which are herein incorporated by reference in its entirety, are known in the art and are suitable for use in the disclosed compositions and methods. In some embodiments, antisense oligonucleotides useful for inhibiting the activity of microRNAs may be about 19 to about 25 nucleotides in length. Antisense oligo nucleotides may include a sequence that is at least partially complementary to a mature miRNA sequence, e.g., at least about 75%, 80%, 85%, 90%, 95%, 96%, 97%, 98%, or 99% complementary to a mature miRNA sequence. In some embodiments, the antisense oligonucleotide may be substantially complementary to a mature miRNA sequence, e.g., at least about 95%, 96%, 97%, 98%, or 99% complementary to a target polynucleotide sequence. In some embodiments, the antisense oligonucleotide comprises a sequence that is 100% complementary to a mature miRNA sequence.

In some embodiments, the antagonist of miR-29a-c is a chemically modified antisense oligo nucleotide. The chemically modified antisense oligonucleotide may comprise a sequence that is at least partially complementary to the mature miRNA sequence of miR-29a, miR-29b, or miR-29c. In some embodiments, the chemically modified antisense oligonucleotide comprises a sequence that is at least partially complementary to the sequence of SEQ ID NO: 1, SEQ ID NO: 2, or SEQ ID NO: 3. In some embodiments, the chemically modified antisense oligonucleotide comprises a sequence that is 100% complementary to SEQ ID NO: 1, SEQ ID NO: 2, or SEQ ID NO: 3.

In some embodiments, antisense oligonucleotides may comprise a sequence that is substantially complementary to a precursor miRNA sequence (pre-miRNA) for miR-29a-c, e.g., at least about 95%, 96%, 97%, 98%, or 99% complementary to a precursor miRNA sequence (pre-miRNA) for miR-29a-c. In some embodiments, the antisense oligonucleotide comprises a sequence that is substantially complementary (e.g., at least about 95%, 96%, 97%, 98%, or 99% complementary) to a sequence located outside the stem-loop region of the pre-miR-29a, pre-miR-29b, or pre-miR-29c sequence.

In some embodiments, the antagonist of miR-29a-c may be an inhibitory RNA molecule having at least partial sequence identity (e.g., about 75%, 80%, 85%, 90%, 95%, 96%, 97%, 98%, or 99%) with the mature miR-29a, miR-29b and miR-29c sequences. The inhibitory RNA molecule may be a double-stranded, small interfering RNA (siRNA) or a short hairpin RNA molecule (shRNA) having a stem-loop structure. The double-stranded regions of the inhibitory RNA molecule may comprise a sequence that is at least partially identical, e.g., about 75%, 80%, 85%, 90%, 95%, 96%, 97%, 98%, or 99% identical to the mature miRNA sequence. In some embodiments, the double stranded regions of the inhibitory RNA comprise a sequence that is at least substantially identical to the mature miRNA sequence. “Substantially identical” refers to a sequence that is about 95%, 96%, 97%, 98%, or 99% identical to a target polynucleotide sequence. In other embodiments, the double stranded regions of the inhibitory RNA molecule may be 100% identical to the target miRNA sequence.

In some embodiments, an antagonist of miR-29a-c is an inhibitory RNA molecule having a double-stranded region, wherein the double-stranded region comprises a sequence having, e.g., 75%, 80%, 85%, 90%, 95%, 96%, 97%, 98%, 99%, or 100% identity with the mature miR-29a (SEQ ID NO: 1), miR-29b (SEQ ID NO: 2), or miR-29c (SEQ ID NO: 3) sequence. In some embodiments, antagonists of miR-29a-c are inhibitory RNA molecules which comprise a double-stranded region, wherein the double-stranded region comprises a sequence of at least about 75%, 80%, 85%, 90%, 95%, 96%, 97%, 98%, or 99% identity to the mature miR-29a, miR-29b, or miR-29c sequence.

In some embodiments, the inhibitory RNA molecule may be a ribozyme. A ribozyme is a catalytic RNA that hydrolyzes phosphodiester bonds of RNA molecules. The ribozyme may be designed to target one or more of miR-29a, miR-29b, and miR-29c resulting in their hydrolysis.

In some embodiments, an antagonist of miR-29a-c may be polynucleotides comprising at least a portion of a complementary sequence of the mature miR-29a-c. In some embodiments, the polynucleotide comprises the complementary sequence of a polynucleotide sequence of SEQ ID NOs: 1-3. In some embodiments, an antagonist of miR-29a-c may be a polynucleotide comprising at least a portion of a complementary sequence of pri-miRNA or pre-miRNA sequence for miR-29a, miR-29b, and/or miR-29c.

In some embodiments, the polynucleotide comprising a complementary sequence of the mature miR-29a-c, pre-miR-29a-c, or pri-miR-29a-c sequence may be single stranded or double stranded.

In some embodiments, the polynucleotide may contain one or more chemical modifications, such as locked nucleic acids, peptide nucleic acids, sugar modifications, 2′-fluoro, and 4′ thio modifications, and backbone modifications, such as one or more phosphorothioate, morpholino, or phosphonocarboxylate linkages.

In some embodiments, the polynucleotide may include a complementary sequence of miR-29a-c that is conjugated to cholesterol.

In some embodiments, an antagonist of miR-29a-c may be an agent distinct from miR-29a-c that acts to decrease, suppress, or prevent the function of miR-29a-c.

In some embodiments, the antagonist may include a polynucleotide sequence of SEQ ID NOs: 4-107, as described in Table 2 below. In some embodiments, the antagonist may include a polynucleotide sequence having at least 80% (e.g., 80%, 85%, 90%, 95%, 99%) sequence identity with a polynucleotide sequence of SEQ ID NOs: 4-107.

TABLE 2
Example miR-29a-c Antagonists (RNA type)

| SEQ | | |
|---|---|---|
| ID | | |
| NO | Sequence | target |
| 4 | UAACCGAUUUCAGAUGGUGCUA | hsa-miR- |
| 5 | UAACCGAUUUCAGAUGGUGCUAs | 29-a |
| 6 | UAACCGAUUUCAGAUGGUGCUsAs | |
| 7 | UAACCGAUUUCAGAUGGUGCsUsAs | |
| 8 | UAACGAUUUCAGAUGGUGsCsUsAs | |
| 9 | UAACCGAUUUCAGAUGGUsGsCsUsAs | |
| 10 | UsAACCGAUUUCAGAUGGUGCUA | |
| 11 | UsAACCGAUUUCAGAUGGUGCUAs | |
| 12 | UsAACCGAUUUCAGAUGGUGCUsAs | |
| 13 | UsAACCGAUUUCAGAUGGUGCsUsAs | |
| 14 | UsAACCGAUUUCAGAUGGUGsCsUsAs | |
| 15 | UsAACCGAUUUCAGAUGGUsGsCsUsAs | |
| 16 | UsAsACCGAUUUCAGAUGGUGCUA | |
| 17 | UsAsACCGAUUUCAGAUGGUGCUAs | |
| 18 | UsAsACCGAUUUCAGAUGGUGCUsAs | |
| 19 | UsAsACCGAUUUCAGAUGGUGCsUsAs | |
| 20 | UsAsACCGAUUUCAGAUGGUGsCsUsAs | |
| 21 | UsAsACCGAUUUCAGAUGGUsGsCsUsAs | |
| 22 | UsAsAsCCGAUUUCAGAUGGUGCUAs | |
| 23 | UsAsAsCCGAUUUCAGAUGGUGCUsAs | |
| 24 | UsAsAsCCGAUUUCAGAUGGUGCsUsAs | |
| 25 | UsAsAsCCGAUUUCAGAUGGUGsCsUsAs | |
| 26 | AACACUGAUUUCAAAUGGUGCUA | hsa-miR- |
| 27 | AACACUGAUUUCAAAUGGUGCUAs | 29-b |
| 28 | AACACUGAUUUCAAAUGGUGCUsAs | |
| 29 | AACACUGAUUUCAAAUGGUGCsUsAs | |
| 30 | AACACUGAUUUCAAAUGGUGsCsUsAs | |
| 31 | AACACUGAUUUCAAAUGGUsGsCsUsAs | |
| 32 | AsACACUGAUUUCAAAUGGUGCUA | |
| 33 | AsACACUGAUUUCAAAUGGUGCUAs | |
| 34 | AsACACUGAUUUCAAAUGGUGCUsAs | |
| 35 | AsACACUGAUUUCAAAUGGUGCsUsAs | |
| 36 | AsACACUGAUUUCAAAUGGUGsCsUsAs | |
| 37 | AsACACUGAUUUCAAAUGGUsGsCsUsAs | |
| 38 | AsAsCACUGAUUUCAAAUGGUGCUA | |
| 39 | AsAsCACUGAUUUCAAAUGGUGCUAs | |
| 40 | AsAsCACUGAUUUCAAAUGGUGCUsAs | |
| 41 | AsAsCACUGAUUUCAAAUGGUGCsUsAs | |
| 42 | AsAsCACUGAUUUCAAAUGGUGsCsUsAs | |
| 43 | AsAsCACUGAUUUCAAAUGGUsGsCsUsAs | |
| 44 | AsAsCsACUGAUUUCAAAUGGUGCUA | |
| 45 | AsAsCsACUGAUUUCAAAUGGUGCUAs | |
| 46 | AsAsCsACUGAUUUCAAAUGGUGCUsAs | |
| 47 | AsAsCsACUGAUUUCAAAUGGUGCsUsAs | |
| 48 | AsAsCsACUGAUUUCAAAUGGUGsCsUsAs | |
| 49 | AsAsCsACUGAUUUCAAAUGGUsGsCsUsAs | |
| 50 | UAACCGAUUUCAAAUGGUGCUA | hsa-miR- |
| 51 | UAACCGAUUUCAAAUGGUGCUAs | 29-c |
| 52 | UAACCGAUUUCAAAUGGUGCUsAs | |
| 53 | UAACCGAUUUCAAAUGGUGCsUsAs | |
| 54 | UAACCGAUUUCAAAUGGUGsCsUsAs | |
| 55 | UAACCGAUUUCAAAUGGUsGsCsUsAs | |
| 56 | UsAACCGAUUUCAAAUGGUGCUA | |
| 57 | UsAACCGAUUUCAAAUGGUGCUAs | |
| 58 | UsAACCGAUUUCAAAUGGUGCUsAs | |
| 59 | UsAACCGAUUUCAAAUGGUGCsUsAs | |
| 60 | UsAACCGAUUUCAAAUGGUGsCsUsAs | |
| 61 | UsAACCGAUUUCAAAUGGUsGsCsUsAs | |
| 62 | UsAsACCGAUUUCAAAUGGUGCUA | |
| 63 | UsAsACCGAUUUCAAAUGGUGCUAs | |
| 64 | UsAsACCGAUUUCAAAUGGUGCUsAs | |
| 65 | UsAsACCGAUUUCAAAUGGUGCsUsAs | |
| 66 | UsAsACCGAUUUCAAAUGGUGsCsUsAs | |
| 67 | UsAsACCGAUUUCAAAUGGUsGsCsUsAs | |
| 68 | UsAsAsCCGAUUUCAAAUGGUGCUA | |
| 69 | UsAsAsCCGAUUUCAAAUGGUGCUAs | |
| 70 | UsAsAsCCGAUUUCAAAUGGUGCUsAs | |
| 71 | UsAsAsCCGAUUUCAAAUGGUGCsUsAs | |
| 72 | UsAsAsCCGAUUUCAAAUGGUGsCsUsAs | |
| 73 | UsAsAsCCGAUUUCAAAUGGUsGsCsUsAs | |
| 74 | AACACCGAUUUCAAAUGGUGCUA | miR-29a-c |
| 75 | AACACCGAUUUCAAAUGGUGCUAs | |
| 76 | AACACCGAUUUCAAAUGGUGCUsAs | |
| 77 | AACACCGAUUUCAAAUGGUGCsUsAs | |
| 78 | AACACCGAUUUCAAAUGGUGsCsUsAs | |
| 79 | AACACCGAUUUCAAAUGGUsGsCsUsAs | |
| 80 | AsACACCGAUUUCAAAUGGUGCUA | |
| 81 | AsACACCGAUUUCAAAUGGUGCUAs | |
| 82 | AsACACCGAUUUCAAAUGGUGCUsAs | |
| 83 | AsACACCGAUUUCAAAUGGUGCsUsAs | |
| 84 | AsACACCGAUUUCAAAUGGUGsCsUsAs | |
| 85 | AsACACCGAUUUCAAAUGGUsGsCsUsAs | |
| 86 | AsAsCACCGAUUUCAAAUGGUGCUA | |
| 87 | AsAsCACCGAUUUCAAAUGGUGCUAs | |
| 88 | AsAsCACCGAUUUCAAAUGGUGCUsAs | |
| 89 | AsAsCACCGAUUUCAAAUGGUGCsUsAs | |
| 90 | AsAsCACCGAUUUCAAAUGGUGsCsUsAs | |
| 91 | AsAsCACCGAUUUCAAAUGGUsGsCsUsAs | |
| 92 | AsAsCsACCGAUUUCAAAUGGUGCUA | |
| 93 | AsAsCsACCGAUUUCAAAUGGUGCUAs | |
| 94 | AsAsCsACCGAUUUCAAAUGGUGCUsAs | |
| 95 | AsAsCsACCGAUUUCAAAUGGUGCsUsAs | |
| 96 | AsAsCsACCGAUUUCAAAUGGUGsCsUsAs | |
| 97 | AsAsCsACCGAUUUCAAAUGGUsGsCsUsAs | |
| 98 | AsAsCsAsCCGAUUUCAAAUGGUGCUAs | |
| 99 | AsAsCsAsCCGAUUUCAAAUGGUGCUsAs | |
| 100 | AsAsCsAsCCGAUUUCAAAUGGUGCsUsAs | |
| 101 | AsAsCsAs CCGAUUUCAAAUGGUGsCsUsAs | |
| 102 | AsAsCsAsCCGAUUUCAAAUGGUsGsCsUsAs | |
| 103 | AsAsCsAsCsCGAUUUCAAAUGGUGCUAs | |
| 104 | AsAsCsAsCsCGAUUUCAAAUGGUGCUsAs | |
| 105 | AsAsCsAsCsCGAUUUCAAAUGGUGCsUsAs | |
| 106 | AsAsCsAsCsCGAUUUCAAAUGGUGsCsUsAs | |
| 107 | AsAsCsAsCsCGAUUUCAAAUGGUsGsCsUsAs | |

s: represents modifications or replacement to the ribonucleotides

As mentioned above, RNA molecules can be used as an antagonist against expression or function of miR-29a-c. The antagonist may be a small RNA molecule similar to the sequences in Table 2 (e.g., having at least 40% identical, including nucleotide position). The antagonist may be a large RNA molecule containing the sequences in Table 2 or similar sequences thereof. The antagonist may be a (chemically) modified RNA molecule containing the sequences in Table 2 or similar sequences thereof. Antagonists may comprise one or more modified nucleotides, such as 2′-O-methyl-sugar modifications. Antagonists may also comprise one or more phosphorothioate linkages resulting in a partial or full phosphorothioate backbone.

In some embodiments, expression vectors may be employed to express an antagonist of miR-29a-c (e.g., antagomirs, antisense oligonucleotides, inhibitory RNA molecules). In some embodiments, an expression vector for expressing an antagonist of miR-29a-c comprises a promoter operably linked to a polynucleotide encoding an antisense oligonucleotide, wherein the sequence of the expressed antisense oligonucleotide is at least partially complementary to the mature miR-29a, miR-29b, or miR-29c sequence. In some embodiments, an expression vector for expressing an inhibitor of miR-29a-c comprises one or more promoters operably linked to a polynucleotide encoding a shRNA or siRNA, wherein the expressed shRNA or siRNA comprises a sequence that is identical, partially identical, or substantially identical to the mature miR-29a, miR-29b, or miR-29c sequence. “Partially identical” refers to a sequence that is at least about 75%, 80%, 85%, 90%, 95%, 96%, 97%, 98%, or 99% identical to a target polynucleotide sequence. “Substantially identical refers to a sequence that is at least about 95%, 96%, 97%, 98%, or 99% identical to a target polynucleotide sequence.

In some embodiments, the expression construct may include naked recombinant DNA or RNA. Transfer of the construct may be performed by any of the methods which physically or chemically permeabilize the cell membrane. This is particularly applicable for transfer in vitro, but it may be applied to in vivo use as well.

In some embodiments, the antagonist of miR-29a-c may be expressed in vivo from a vector. A “vector” refers to a composition of matter which can be used to deliver a nucleic acid of interest to the interior of a cell. Numerous vectors are known in the art including, but not limited to, linear poly nucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. An expression construct can be replicated in a living cell, or it can be made synthetically. The terms “expression construct,” “expression vector,” and “vector” are used interchangeably in this disclosure.

In some embodiments, an expression vector for expressing antagonists of miR-29a-c comprises a promoter operably linked to a polynucleotide encoding an antagonist of miR-29a, miR-29b, miR-29c, or combinations thereof. In some embodiments, the polynucleotide may encode an antagonist of the miR-29b-1/miR-29a cluster. In some embodiments, the polynucleotide may encode the antagonist of the miR-29b-2/miR-29c cluster.

The term “operably linked’ or “under transcriptional control,” as used herein, refers to that the promoter is in the correct location and orientation in relation to a polynucleotide to control the initiation of transcription by RNA polymerase and expression of the polynucleotide.

In some embodiments, the polynucleotide encoding an antagonist of miR-29a-c may encode a complementary sequence of primary-microRNA-29a-c sequence (pri-miR-29a-c), the precursor-microRNA-29a-c sequence (pre-miR-229a-c) or the mature miR-29a-c sequence.

In some embodiments, the expression vector may include a polynucleotide operably linked to a promoter. In some embodiments, the polynucleotide may include a complementary sequence of SEQ ID NO: 1. In some embodiments, the expression vector may include a polynucleotide operably linked to a promoter. In some embodiments, the polynucleotide may include a complementary sequence of SEQ ID NO: 2. In some embodiments, the expression vector may include a polynucleotide operably linked to a promoter. In some embodiments, the polynucleotide may include a complementary sequence of SEQ ID NO: 3.

In some embodiments, the polynucleotide comprising a complementary sequence of SEQ ID NO: 1, SEQ ID NO: 2, or SEQ ID NO: 3 may be about 18 to about 2000 nucleotides in length, e.g., about 70 to about 200 nucleotides in length, about 20 to about 50 nucleotides in length, or about 18 to about 25 nucleotides in length.

As used herein, “expression construct” refers to any type of genetic construct containing a nucleic acid coding for a gene product in which part or all the nucleic acid encoding sequence is capable of being transcribed. Generally, the nucleic acid encoding a gene product is under transcriptional control of a promoter.

As used herein, “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for an RNA polymerase. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 by of DNA, and containing one or more recognition sites for transcriptional activator or repressor proteins. At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 by apart before activity begins to decline. Depending on the promoter, it appears that individual elements can function either co-operatively or independently to activate transcription.

In other embodiments, the human cytomegalovirus (CMV) immediate early gene promoter, the SV40 early promoter, the Rous sarcoma virus long terminal repeat, rat insulin promoter, RNA pol III promoter, and glyceraldehyde-3-phosphate dehydrogenase promoter can be used to obtain high-level expression of the polynucleotide of interest. The use of other viral or mammalian cellular or bacterial phage promoters which are well-known in the art to achieve expression of a polynucleotide of interest is contemplated as well, provided that the levels of expression are sufficient for a given purpose.

By employing a promoter with well-known proper ties, the level and pattern of expression of the polynucleotide of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Viral promoters, cellular promoters/enhancers, and inducible promoters/enhancers that could be used in combination with the nucleic acid encoding a gene of interest in an expression construct are described, e.g., in U.S. Pat. No. 8,440,636, which is hereby incorporated by reference. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct. In some embodiments, the polynucleotide encoding the miR-29a-c antagonist is operably linked to a fibroblast-specific promoter.

As will be further described in the next section of this disclosure, in some embodiments, the expression construct may be entrapped in a liposome. Liposomes are vesicular structures characterized by a phospholipid bilayer membrane and an inner aqueous medium. Multilamellar liposomes have multiple lipid layers separated by an aqueous medium. They form spontaneously when phospholipids are suspended in excess of an aqueous solution. The lipid components undergo self-rearrangement before the formation of closed structures and entrap water and dissolved solutes between the lipid bilayers (Ghosh and Bachhawat, Glycobiology. 1991: 1(5):505-10). Also within the scope of this disclosure are lipofectamine-DNA complexes.

In addition to collagen generation, also within the scope of this disclosure are the agents (e.g., nucleic acid therapeutics) targeting other functions, including antioxidant response, extracellular matrix (ECM) integrity maintenance, and hydration.

Skin is a very important organ that is frequently affected by oxidative stress, caused by either intrinsic or external factors, such as aging or UV damage. Oxidative stress plays an important role in the development of skin aging, including wrinkles, pigmented spots, and skin diseases such as cancer. By increasing the level of the mRNA of the genes (e.g., CAT, SOD1, SOD2, GPx1, and GPx4), one can increase the expression of catalase, superoxide dismutase, and glutathione peroxidase, thereby enhancing the antioxidant defense of the skin. In one example, multiple mRNA molecules may be directly or indirectly linked, with each mRNA targeting one of the following genes, to enhance the anti-oxidation effect. In some embodiments, the agents may improve skin conditions or treat skin diseases or disorders by:
(1) enhancing antioxidant/response to stress gene, including but not limited to, CAT: catalase; GPX1: glutathione peroxidase 1; SOD1: superoxide dismutase 1; and SOD 2: superoxide dismutase 2, mitochondrial.

Collagen and elastin, both in the dermis, retain skin elasticity and flexibility; Matrix metalloproteinases (MMPs) degrade extracellular matrix (ECM) proteins, such as collagen and elastin. In addition to collagen and elastin, hyaluronic acid is abundant in the dermis; it retains moisture and thus determines the moisture content of the skin. Hyaluronic acid is synthesized by hyaluronic acid synthase (HAS).

By enhancing the mRNA levels of genes related to the extracellular matrix (ECM) (such as COL1A1, COL3A1, ELN, HAS2 and HAS3), and that of the degrading enzyme, MMP1, and MMP2, the expression of ECM proteins and enzymes involved in ECM turnover can be enhanced.
(2) inhibiting extracellular matrix breakdown using siRNA linkage, with each siRNA molecule targeting the genes, including MMP1: matrix metallopeptidase 1; and MMP2: matrix metallopeptidase 2.
(3) enhancing ECM integrity using mRNA linkage, with each mRNA molecule targeting the following genes: COL1A1: collagen, type I, alpha 1; COL3A1: collagen, typeIII, alpha 1; and ELN: elastin.
(4) enhancing hydration gene using mRNA linkage, with each mRNA molecule targeting the following genes: AQP3: aquaporin3; and HAS2: hyaluronic acid synthase 2.
(5) targeting TYR, TRP1, and MITF as therapeutic targets for depigmentation; and
(6) targeting the key molecules in p53/p21 or p16/Rb pathways to regulate the redox-sensitive kinases ERK1/2 and p38, and to inhibit the cylcine-dependent kinases that are significant in regulating skin cell senescence,

The agents may include a group of small interfering RNA fragments associated with microRNA 29 (also referred to as miR-29 or miR-29). Delivery systems may be lipid-based, surfactant-based, polymers-based, peptide-based, or a combination thereof.

b. Transdermal Delivery Systems

In some embodiments, the composition may include a transdermal delivery system (or a carrier) for dermal delivery of the disclosed agents (e.g., nucleic acid therapeutics).

In some embodiments, the transdermal delivery system can be lipid-based, surfactant-based, polymers-based, peptide-based, or a combination thereof.

Non-limiting examples of the transdermal delivery system that can be used in the context of this disclosure for transdermal delivery of miR-29a-c antagonists may include the following example formulations.

### 1. Liposome Transdermal Delivery System

In some embodiments, the liposome transdermal delivery system may include phospholipids, cholesterol, and PEG [N-(carbamoyl-methoxypolyethylene glycol XXX)-1, 2-distearoyl-sn-glycero-3-phosphoethanolamine sodium salt].

In some embodiments, phospholipids may be saturated or unsaturated. In some embodiments, phospholipids may have a carbon chain with 16-22 (e.g., 16, 17, 18, 19, 20, 21, 22) carbons. In some embodiments, phospholipids may include hydrogenated soy phosphatidylcholine (HSPC), distearoylphosphatidylcholine (DSPC), 1,2-dioleoyl-sn-glycero-3-phosphocholine (DOPC), 1,2-distearoyl-3-sn-glycerophosphoethanolamine (DSPE), dioleoylphosphatidylethanolamine (DOPE), or a combination thereof.

In some embodiments, glycol XXX can be glycol having a molecular weight of 120-5000 Daltons (e.g., 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 Daltons).

In some embodiments, the liposome deliver system include 40-90 wt % (e.g., 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %) of phospholipid, 10-60 wt % (e.g., 10 wt %, 15 wt %, 20 wt %, 25 wt %, 30 wt %, 35 wt %, 40 wt %, 45 wt %, 50 wt %, 55 wt %, 60 wt %) of cholesterol, and 0-7 wt % (e.g., 1 wt %, 2 wt %, 3 wt %, 4 wt %, 5 wt %, 6 wt %, 7 wt %) of PEG.

### 2. Cationic Liposome Transdermal Delivery System

(1) The above delivery system “1” with cationic lipids

Non-limiting examples of cationic lipids may include DOTAP (N-[1-(2,3-dioleoloxy)propyl]-N,N,N-trimethyl ammonium chloride), DDAB [Dimethyldioctadecylammonium (Bromide Salt)], or a combination thereof. In some embodiments, the cationic liposome transdermal delivery system may include 0-55 wt % (e.g., 5 wt %, 10 wt %, 15 wt %, 20 wt %, 25 wt %, 30 wt %, 35 wt %, 40 wt %, 45 wt %, 50 wt %, 55 wt %) of cationic lipids.
(2) The cationic liposome transdermal delivery system containing cholesterol and cationic lipid

In some embodiments, the cationic liposome transdermal delivery system may include about 10-60 wt % (e.g., 10 wt %, 15 wt %, 20 wt %, 25 wt %, 30 wt %, 35 wt %, 40 wt %, 45 wt %, 50 wt %, 55 wt %, 60 wt %) of cholesterol and about 40-90 wt % (e.g., 40 wt %, 45 wt %, 50 wt %, 55 wt %, 60 wt %, 65 wt %, 70 wt %, 75 wt %, 80 wt %, 85 wt %, 90 wt %) of cationic lipid.
(3) The cationic liposome transdermal delivery system containing cholesterol, cationic lipid, and PEG

In some embodiments, the cationic liposome transdermal delivery system may include about 0-8 wt % (e.g., 1 wt %, 2 wt %, 3 wt %, 4 wt %, 5 wt %, 6 wt %, 7 wt %, 8 wt %) of PEG.
(4) The cationic liposome transdermal delivery system containing phospholipid and cationic lipid.

In some embodiments, the cationic liposome transdermal delivery system may include 40-60 wt % (e.g., 40 wt %, 45 wt %, 50 wt %, 55 wt %, 60 wt %) of phospholipid, and about 40-60 wt % (e.g., 40 wt %, 45 wt %, 50 wt %, 55 wt %, 60 wt %) of cationic lipid.
(5) The cationic liposome transdermal delivery system containing phospholipid, cationic lipid, and PEG

In some embodiments, the cationic liposome transdermal delivery system may include 40-60 wt % (e.g., 40 wt %, 45 wt %, 50 wt %, 55 wt %, 60 wt %) of phospholipid, about 40-60 wt % (e.g., 40 wt %, 45 wt %, 50 wt %, 55 wt %, 60 wt %) of cationic lipid, and about 0-8 wt % (e.g., 1 wt %, 2 wt %, 3 wt %, 4 wt %, 5 wt %, 6 wt %, 7 wt %, 8 wt %) of PEG.
(6) The cationic liposome transdermal delivery system containing cationic lipid and PEG

In some embodiments, the cationic liposome transdermal delivery system may include 92-99 wt % (e.g., 92 wt %, 93 wt %, 94 wt %, 95 wt %, 96 wt %, 97 wt %, 98 wt %, 99 wt %) of cationic lipid and about 0-8 wt % (e.g., 1 wt %, 2 wt %, 3 wt %, 4 wt %, 5 wt %, 6 wt %, 7 wt %, 8 wt %) of PEG.

3. pH-Dependent Cationic Liposomes, Composed of
(1) The above delivery system “1” with pH-dependent cationic lipids

Non-limiting examples of pH-dependent cationic lipids may include 1,2-dioleoyl-3-dimethylammonium-propane (DODAP), N-palmitoyl homocysteine (PHC), multivalent cationic lipids, or a combination thereof.

pH-dependent cationic lipids are able to encapsulate nucleic acids and form complexes at low pH. The surface potential of the complex system is neutral at physiological pH, and predominantly positively charged at low pH, which facilitates the interaction of the complex with lysosomal membrane and the release of nucleic acid content into the cytoplasm.

In some embodiments, the pH-dependent cationic liposome transdermal delivery system may include about 0-55 wt % (e.g., 5 wt %, 10 wt %, 15 wt %, 20 wt %, 25 wt %, 30 wt %, 35 wt %, 40 wt %, 45 wt %, 50 wt %, 55 wt %) of pH-dependent cationic lipid.
(2) The pH-dependent cationic liposome transdermal delivery system containing phospholipid and pH-dependent cationic lipid.

In some embodiments, the pH-dependent cationic liposome transdermal delivery system may include about 0-55 wt % (e.g., 5 wt %, 10 wt %, 15 wt %, 20 wt %, 25 wt %, 30 wt %, 35 wt %, 40 wt %, 45 wt %, 50 wt %, 55 wt %) of pH-dependent cationic lipid, about 45-95 wt % (e.g., 45 wt %, 50 wt %, 55 wt %, 60 wt %, 65 wt %, 70 wt %, 75 wt %, 80 wt %, 85 wt %, 90 wt %, 95 wt %) of phospholipid.
(3) The pH-dependent cationic liposome transdermal delivery system containing cholesterol and pH-dependent cationic lipids

In some embodiments, the pH-dependent cationic liposome transdermal delivery system may include about 10-60 wt % (e.g., 10 wt %, 15 wt %, 20 wt %, 25 wt %, 30 wt %, 35 wt %, 40 wt %, 45 wt %, 50 wt %, 55 wt %, 60 wt %) of cholesterol and about 40-90 wt % (e.g., 40 wt %, 45 wt %, 50 wt %, 55 wt %, 60 wt %, 65 wt %, 70 wt %, 75 wt %, 80 wt %, 85 wt %, 90 wt %) of pH-dependent cationic lipid.
(4) The pH-dependent cationic liposome transdermal delivery system containing phospholipid, cholesterol, cationic lipid, and pH-dependent cationic lipid

In some embodiments, the pH-dependent cationic liposome deliver system may include 40-90 wt % (e.g., 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %) of phospholipid, 10-60 wt % (e.g., 10 wt %, 15 wt %, 20 wt %, 25 wt %, 30 wt %, 35 wt %, 40 wt %, 45 wt %, 50 wt %, 55 wt %, 60 wt %) of cholesterol, about 40-90 wt % (e.g., 40 wt %, 45 wt %, 50 wt %, 55 wt %, 60 wt %, 65 wt %, 70 wt %, 75 wt %, 80 wt %, 85 wt %, 90 wt %) of cationic lipid, and about 0-55 wt % (e.g., 5 wt %, 10 wt %, 15 wt %, 20 wt %, 25 wt %, 30 wt %, 35 wt %, 40 wt %, 45 wt %, 50 wt %, 55 wt %) of pH-dependent cationic lipid.
(5) The pH-dependent cationic liposome transdermal delivery system containing phospholipid, cationic lipid, and pH-dependent cationic lipid

In some embodiments, the pH-dependent cationic liposome deliver system may include 40-90 wt % (e.g., 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %) of phospholipid, about 40-90 wt % (e.g., 40 wt %, 45 wt %, 50 wt %, 55 wt %, 60 wt %, 65 wt %, 70 wt %, 75 wt %, 80 wt %, 85 wt %, 90 wt %) of cationic lipid, and about 0-55 wt % (e.g., 5 wt %, 10 wt %, 15 wt %, 20 wt %, 25 wt %, 30 wt %, 35 wt %, 40 wt %, 45 wt %, 50 wt %, 55 wt %) of pH-dependent cationic lipid.
(6) The pH-dependent cationic liposome transdermal delivery system containing cholesterol, cationic lipid, and pH-dependent cationic lipid

In some embodiments, the pH-dependent cationic liposome deliver system may include 10-60 wt % (e.g., 10 wt %, 15 wt %, 20 wt %, 25 wt %, 30 wt %, 35 wt %, 40 wt %, 45 wt %, 50 wt %, 55 wt %, 60 wt %) of cholesterol, about 40-90 wt % (e.g., 40 wt %, 45 wt %, 50 wt %, 55 wt %, 60 wt %, 65 wt %, 70 wt %, 75 wt %, 80 wt %, 85 wt %, 90 wt %) of cationic lipid, and about 0-55 wt % (e.g., 5 wt %, 10 wt %, 15 wt %, 20 wt %, 25 wt %, 30 wt %, 35 wt %, 40 wt %, 45 wt %, 50 wt %, 55 wt %) of pH-dependent cationic lipid.
(7) The pH-dependent cationic liposome transdermal delivery system containing pH-dependent cationic lipid

In some embodiments, the pH-dependent cationic liposome deliver system may include about 45-90 wt % (e.g., 45 wt %, 50 wt %, 55 wt %, 60 wt %, 65 wt %, 70 wt %, 75 wt %, 80 wt %, 85 wt %, 90 wt %) of cationic lipid, and about 10-55 wt % (e.g., 10 wt %, 15 wt %, 20 wt %, 25 wt %, 30 wt %, 35 wt %, 40 wt %, 45 wt %, 50 wt %, 55 wt %) of pH-dependent cationic lipid.
(8) (2) to (7) with PEG or PEG derivatives

In some embodiments, the pH-dependent cationic liposome deliver system may include about 0-8 wt % (e.g., 1 wt %, 2 wt %, 3 wt %, 4 wt %, 5 wt %, 6 wt %, 7 wt %, 8 wt %) of PEG or PEG derivatives.

4. Liposome Transdermal Delivery System with Specific Edge Activators or Inorganic Particles

In some embodiments, the liposome transdermal delivery system may include the above delivery systems “1,” “2,” and “3” and edge activators or inorganic particles.

In some embodiments, the liposome transdermal delivery system may include 90-99 wt % (e.g., 90 wt %, 91 wt %, 92 wt %, 93 wt %, 94 wt %, 95 wt %, 96 wt %, 97 wt %, 98 wt %, 99 wt %) of the above delivery system “1,” “2,” or “3”, and about 0-10 wt % (e.g., 1 wt %, 2 wt %, 3 wt %, 4 wt %, 5 wt %, 6 wt %, 7 wt %, 8 wt %, 9 wt %, 10 wt %) of edge activators or inorganic particles.

In some embodiments, the edge activators or inorganic particles may promote lipid-mediated nucleic acid expression.

In some embodiments, the edge activator and inorganic particle may include sodium cholate, Span, Tween, and carbonate apatite.

### 5. Ethosome Transdermal Delivery System

In some embodiments, the liposome transdermal delivery system may include the above delivery systems “1,” “2,” “3,” and “4” and a skin penetration enhancer.

In some embodiments, the liposome transdermal delivery system may include about 20-45 wt % (e.g., 25 wt %, 30 wt %, 35 wt %, 40 wt %, 45 wt %) of the skin penetration enhancer. In some embodiments, the skin penetration enhancer may include ethanol.

### 6. Niosome Transdermal Delivery System

Niosomes are uni/bi/multilamellar vesicles formed by self-assembly of hydrated nonionic surfactants, with or without incorporation of cholesterol or their lipids. Niosomes are suitable for delivery of both hydrophobic as well as hydrophilic compounds. Niosomes are used in cosmetics, and skin care applications since skin penetration of ingredients is enhanced because it possesses the property of reversibly reducing the barrier resistance of the homy layer, allowing the ingredient to reach the living tissues at a higher rate.

In some embodiments, the nonionic surfactant may include about 0-20 wt % (e.g., 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt % 17 wt %, 18 wt %, 19 wt %, 20 wt %) of Span, Tween, brijs, alkyl amides, sorbitan ester, crown ester, or polyoxyethylene alkyl ether.

### 7. The Transdermal Delivery System Containing Positively Charged Polymers

Non-limiting examples of positively charged polymers may include:
(1) Diethylaminoethylen—Dextran (DEAE-Dextran)
DEAE-Dextran is a positively charged polysaccharide
(2) Linear and branched polyethylenimine (PEI) and PEI derivatives
(3) Poly(dl-lactide-co-glycolide) [PLGA]
PLGA is a copolymer of glycolic acid (GA) and lactic acid (LA) linked together through ester linkages
(4) Chitosan and modified Chitoson
(5) β-Cyclodextrin
(6) Polypeptides
A. poly{N-[N-(2-aminoethyl)-2-aminoethyl]aspartamide}[PAsp(DET)]
B. Polylysine partially substituted with histidyl residues
At pH below 6.0, the imidazole groups of histidyl residues become protonated, thus conferring a PEI-like proton sponge activity to trigger endosomal escape and demonstrate significant enhancement in transfection compared with polylysine alone.
C. linear cationic amphipathic histidine-rich peptides and derivatives thereof
One example is KKALLALALHHLAHLALHLALALKKA (SEQ ID NO: 108)

These peptides possess high endosomal disruption ability.
(7) Dendrimers
A. PAMAM: Poly(amidoamine) and its derivatives
B. PPI: Poly(propylenimine)

Dendrimers are highly branched monodisperse, highly symmetric, and spherical synthetic macromolecules having tunable structure, size, and surface charge. The structural features, such as high chemical and structural homogeneity, high ligand, and functionality density, enable them to load nucleic acids by interior encapsulation, surface adsorption, or chemical conjugation.

### 8. The Transdermal Delivery System Containing Cell-Penetrating Peptides

In some embodiments, the transdermal delivery system may include cell-penetrating peptides (CPPs) and the delivery systems 1-7 described above

CPPs, also known as protein transduction domains (PTDs), are short peptides containing 5-35 amino acids. They have the ability to penetrate the skin and cross the cell membrane. Thus, CPPs themself and delivery systems 1-7 mentioned above with CPPs are applicable to transdermal delivery of nucleic acids. Non-limiting examples of CPPs may include Tat analog (Tat): GRKKRRQRRRCG (SEQ ID NO: 109) and MPG:

(SEQ ID NO: 110)
GALFLGFLGAAGSTMGAWSQPKKKRKV

### 9. Supplement of the Above Delivery Systems 1-8

Various components may be added to the delivery systems 1-8 described above for accelerated delivery of nucleic acids, such as:
a. Positively charged polycation

They are able to condense nucleic acids and make them encapsulated by liposomes or other delivery systems. In some embodiments, the positively charged polycation may include histone or protamine.
b. Targeting ligands

Various ligand molecules can be added to the delivery systems mentioned above for targeted delivery of nucleic acids. Examples are as followings:
(a) fibroblast growth factor and fibronectin. Fibronectin enhances nucleic acid uptake because of recognition by the extracellular domains of specific molecules on the cell membrane.
(b) synthetic analogs of Luteinizing hormone-releasing hormone targeting peptide.

It is introduced to different dendrimers and expected to enhance the intracellular delivery of siRNA by a receptor-mediated endocytosis pathway.

### C. Compositions

In some embodiments, the above-described agents and the delivery system may be incorporated into a composition (including cosmetic preparations). In some embodiments, the composition may include from about 0.00001 to 100%, such as from 0.001 to 10% or from 0.1% to 5% by weight of one or more agents described herein.

In some embodiments, a disclosed agent (e.g., a nucleic acid therapeutic) described herein may be incorporated into a topical formulation containing a topical earner that is generally suited to topical drug administration and comprising any such material known in the art. The topical carrier may be selected to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols, and other nontoxic organic solvents, glycerin, mineral oil, silicone, petroleumjelly, lanolin, fatty acids, vegetable oils, parabens, waxes, and the like. Formulations may be colorless, odorless ointments, lotions, creams, microemulsions, and gels.

The disclosed agents (e.g., nucleic acid therapeutics) may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

The disclosed agents may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with hydrophilic petrolatum such as that which may be obtained under the trademark Aquaphor™ from Beiersdorf, Inc. (Norwalk, Conn.).

The disclosed agents (e.g., nucleic acid therapeutics) may be incorporated into creams, which generally are viscous liquid or semisolid emulsions, either oil-in-water or water-in-oil. Cream bases are water-washable, and contain an oil phase, an emulsifier, and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

The disclosed agents (e.g., nucleic acid therapeutics) may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase, and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of poly glycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The disclosed agents (e.g., nucleic acid therapeutics) may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose, and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients, and preservatives. An optimum topical formulation comprises approximately: 2 wt % to 60 wt %, preferably 2 wt % to 50 wt %, solubilizer and/or skin permeation enhancer; 2 wt % to 50 wt %, preferably 2 wt % to 20 wt %, emulsifiers; 2 wt % to 20 wt % emollient; and 0.01 to 0.2 wt % preservative, with the active agent and carrier (e.g., water) making of the remainder of the formulation. A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub.1O MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C.sub.2-C.sub.6 alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark AzoneR™ from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations, including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate). In certain topical formulations, the active agent is present in an amount in the range of approximately 0.25 wt % to 75 wt % of the formulation, e.g., in the range of approximately 0.25 wt % to 30 wt % of the formulation, in the range of approximately 0.5 wt % to 15 wt % of the formulation, or in the range of approximately 1.0 wt % to 10 wt % of the formulation. Topical skin treatment compositions can be packaged in a suitable container to suit their viscosity and intended use by the consumer. For example, a lotion or cream can be packaged in a bottle or a roll-ball applicator, a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition.

In some embodiments, colloidal dispersion systems, such as macromolecule complexes, nanocapsules, microspheres, beads, and lipid-based systems, including oil-in-water emulsions, micelles, mixed micelles, and liposomes, may be used as delivery vehicles for the oligonucleotide inhibitors (e.g., antagonists) of microRNA function or constructs expressing particular microRNAs. Commercially available fat emulsions that are suitable for delivering the nucleic acids of the invention to skin fibroblasts include Intralipid R, Liposyn R, Liposyn R. II, LiposynR III, Nutrilipid, and other similar lipid emulsions. A colloidal system for use as a delivery vehicle in vivo may be a liposome (i.e., an artificial membrane vesicle). The preparation and use of such systems is well known in the art. Exemplary formulations are also disclosed in U.S. Pat. Nos. 5,981,505; 6,217,900; 6,383,512; 5,783,565; 7,202,227; 6,379,965; 6,127,170; 5,837,533: U.S. Pat. No. 6,747,014; and WO03/093449, which are herein incorporated by reference in their entireties.

In some embodiments, cosmetic formulations for increasing collagen deposition in tissues may comprise at least one antagonist of miR-29a-c. The antagonist may be an antagonist of miR-29a, miR-29b, miR-29c, or combinations thereof. In some embodiments, the antagonist of miR-29a-c is an antagomir. The antagonist may be linked or conjugated to agents that facilitate the entry of the antagonist into cells or tissues. Such agents may include cell internalization transporters, such as antennapedia, TAT, Buforin II, Transportan, model amphipathic peptide, K-FGF, Ku70, Prion, pVEC, Pep-1, SynB1, SynB3, SynB5, Pep-7, HN-1, Bis-Guanidinium-Spermidine Cholesterol, Bis-Guanidinium-Tren-Cholesterol, and pol yarginine. The agent may be linked to the miR-29a-c antagonist at its amino or carboxy terminus. In one embodiment, the agent is linked to the antagonist by a sequence that is cleaved upon entry to the cell. Such sequences typically comprise consensus sequences for proteases, as are known in the art.

In some embodiments, the cosmetic compositions can be formulated into all types of vehicles. Non-limiting examples of suitable vehicles include emulsions (e.g., water-in-oil, water-in-oil-in-water, oil-in-water, oil-in-water-in-oil, oil-in-water-in silicone emulsions), creams, lotions, Solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in this disclosure. In some embodiments, the concentrations and combinations of the ingredients are selected in such a way that the combinations are chemically compatible and do not form complexes that precipitate from the finished product.

It is also contemplated that aromatic skin-active ingredients and additional ingredients identified throughout this specification can be encapsulated for delivery to a target area such as skin. Non-limiting examples of encapsulation techniques include the use of liposomes, vesicles, and/or nanoparticles (e.g., biodegradable and non-biodegradable colloidal particles comprising polymeric materials in which the ingredient is trapped, encapsulated, and/or absorbed—examples include nanospheres and nanocapsules) that can be used as delivery vehicles to deliver such ingredients to skin (see, e.g., U.S. Pat. No. 6,387,398: U.S. Pat. No. 6,203,802: U.S. Pat. No. 5,411,744; and Kreuter 1998, which are herein incorporated by reference in their entireties).

In some embodiments, the compositions may be pharmaceutically acceptable or pharmacologically acceptable compositions. The phrase “pharmaceutically-acceptable” or “pharmacologically-acceptable” includes compositions that do not produce an allergic or similar untoward reaction when administered to a human. Typically, such compositions are prepared either as topical compositions, liquid solutions or suspensions, solid forms suitable for solution in, or suspension in, liquid prior to use can also be prepared. Routes of administration can vary with the location and nature of the condition to be treated, and include, e.g., topical, inhalation, intradermal, transdermal, parenteral, intravenous, intramuscular, intranasal, subcutaneous, percutaneous, intratracheal, intraperitoneal, intratumoral, perfusion, lavage, direct injection, and oral administration and formulation.

In some embodiments, the compositions may be incorporated into products. Non-limiting examples of products include cosmetic products, food-based products, pharmaceutical products, etc. By way of example only, non-limiting cosmetic products include sunscreen products, sunless skin tanning products, hair products, fingernail products, moisturizing creams, skin benefit creams and lotions, softeners, day lotions, gels, ointments, foundations, night creams, lipsticks, mascaras, eyeshadows, eyeliners, cheek colors, cleansers, toners, masks, or other known cosmetic products or applications. Additionally, cosmetic products can be formulated as leave-on or rinse-off products.

In some embodiments, the compositions may include a topical formulation that can be used in cosmetics. In some embodiments, the cosmetics may be used to prevent or treat skin conditions, including skin aging, alopecia, and scar.

In some embodiments, the composition may be formulated as a non-transdermal system, e.g., used as an injectable for subcutaneous injection for plastic surgery such as wrinkle filler or applied directly into the wounds, which can be surgery incisions or chronic wounds such as diabetic ulcers, etc.

In some embodiments, the composition may be incorporated into a kit or a device (e.g., an applicator). In some embodiments, the composition may be incorporated into a medical device, such as an implantable medical device, including (a) a suture that is used in wound closure to reduce scars, prevent adhesion, reduce inflammation, etc.; (b) wound management patches for chronic wounds, such as diabetic foot ulcer; (c) a barbed suture for minimum facial lifting to enhance and elongate the lifting effect, (d) injectable materials or implant device to increase facial volume or reduce wrinkles or creases, (e) wound closure strip, porous surgical tape strips which can be applied across the laceration or small wound in a manner which pulls the skin on either side of the wound together, and (f) surgical glue, also called “tissue adhesive” or “liquid stitches” to close both major and minor wounds, such as lacerations, incisions made during laparoscopic surgery, and wounds on the face or in the groin. The above medical devices can be surface modified, for example, plasma treatment or coating with another material, to enhance the affinity of the medical device with nucleic acids formulations.

In some embodiments, the compositions may include additional ingredients. Non-limiting examples of additional ingredients include cosmetic ingredients (both active and non-active) and pharmaceutical ingredients (both active and non-active). The CTFA International Cosmetic Ingredient Dictionary and Handbook (2004) describes a wide variety of non-limiting cosmetic ingredients that can be used in the context of this disclosure. Examples of these ingredient classes include: fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, emulsifiers, stabilizers, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraaminobenzoic acid (PABA) and corresponding PABA derivatives, titanium dioxide, Zinc oxide, etc.), essential oils, vitamins (e.g., A, B, C, D, E, and K), trace metals (e.g., Zinc, calcium and Selenium), anti-irritants (e.g., Steroids and non-steroidal anti inflammatories), botanical extracts (e.g., aloe Vera, chamomile, cucumber extract, Ginkgo biloba, Ginseng, and rosemary), anti-microbial agents, antioxidants (e.g., BHT and tocopherol), chelating agents (e.g., disodium EDTA and tetrasodium EDTA), preservatives (e.g. methylparaben and propylparaben), pH adjusters (e.g., sodium hydroxide and citric acid), absorbents (e.g., aluminum starch octenylsuccinate, kaolin, corn starch, oat starch, cyclodextrin, talc, and Zeolite), skin bleaching and lightening agents (e.g., hydro quinone and niacinamide lactate), humectants (e.g., glycerin, propylene glycol, butylene glycol, pentylene glycol, Sorbitol, urea, and manitol), exfoliants (e.g., alpha-hydroxyacids, and beta-hydroxyacids Such as lactic acid, glycolic acid, and Salicylic acid; and salts thereof) waterproofing agents (e.g., magnesium/aluminum hydroxide Stearate), skin conditioning agents (e.g., aloe extracts, allantoin, bisabolol, ceramides, dimethicone, hyaluronic acid, and dipotassium glycyrrhizate), thickening agents (e.g., Substances which that can increase the viscosity of a composition Such as carboxylic acid polymers, crosslinked polyacrylate polymers, polyacrylamide polymers, polysaccharides, and gums), and silicone containing compounds (e.g., silicone oils and polyorganosiloxanes).

In some embodiments, the additional ingredients may include an anti-inflammatory agent or an antibiotic. In some embodiments, the additional ingredients may include topical vitamin A, topical vitamin C, vitamin E, or a combination thereof.

In some embodiments, the compositions may include pharmaceutical ingredients that are useful with the emulsion compositions. Non-limiting examples of pharmaceutical ingredients include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, Scabicides, pediculicides, antineoplastics, antiperspirants, antipruritics, antipsoriatic agents, anti-seborrheic agents, biologically active proteins and peptides, bum treatment agents, cauterizing agents, depigmenting agents, depilatories, diaper rash treatment agents, enzymes, hair growth stimulants, hair growth retardants including DFMO and its salts and analogs, hemostatics, keratolytic, canker Sore treatment agents, cold Sore treatment agents, dental and periodontal treatment agents, photosensitizing actives, skin protectant/barrier agents, steroids including hormones and corticosteroids, Sunburn treatment agents, sunscreens, transdermal actives, nasal actives, vaginal actives, wart treatment agents, wound treatment agents, wound healing agents, etc.

In another aspect, this disclosure provides a kit or device comprising the composition as described herein. The kit may further include water and hybridization buffer to facilitate hybridization of the two strands of the miRNAs. In some embodiments, the kit may include one or more oligonucleotides for inhibiting the function of a target miRNA. The kit may also include one or more transfection reagent(s) to facilitate delivery of the miRNA or miRNA antagonists to cells.

The container means of the kits will generally include at least one vial, test tube, flask, bottle, or other container means, into which a component may be placed and suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of this disclosure also will typically include a means for containing the nucleic acids, and any other reagent containers in close confinement for commercial sale. Such containers may include injection or blow-molded plastic containers into which the desired vials are retained.

In some embodiments, the kit includes also includes an additional therapeutic agent. For example, the kit includes a first container that contains the composition and a second container for the additional therapeutic agent.

In some embodiments, the kit may include informational material of the kits that is not limited in its form. In one embodiment, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

In addition to the composition, the kit can include other ingredients, such as a solvent or buffer, a stabilizer, or a preservative. The composition can be provided in any form, e.g. liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit.

The kit optionally includes a device suitable for application or administration of the composition, e.g., a syringe, a skin adhesive applicator or other suitable delivery device. The device can be provided pre-loaded with one or both of the agents or can be empty, but suitable for loading.

### Methods of Improving Conditions Associated with Collagen Deficiency or Treating Diseases or Disorders Associated with Collagen Deficiency

The agents, formulations, products and methods disclosed herein can be used for improving conditions associated with collagen deficiency or for treating diseases or disorders associated with collagen deficiency in a subject. The subject may be suffering from or be predisposed to a condition characterized by, or associated with, a collagen deficiency, collagen malfunction, or a connective tissue related condition benefiting from an augmentation of collagen in the connective tissue, including skin wounds or lesions, or a connective tissue disease or injury.

Collagen is the most abundant protein in the body. Collagen deficiency means that the body does not have an adequate supply of collagen. Severe collagen deficiency is rare; however, even a minor deficiency can cause symptoms that interfere with the quality of life and make daily activities difficult. Conditions characterized by, or associated with collagen deficiency include, but are not limited to conditions in which the biosynthesis, assembly, posttranslational modification and/or secretion of collagen is affected, often due to an underlying genetic defect. Such conditions include skin disorders or bad appearance, hair disorders or bad appearance, nail disorder bad appearance, bone disorders, joint disorders, connective tissue disorders (e.g. collagenopathies), muscle disorders (myopathies), and disorders of basement membrane disorders. Collagen deficiency can lead to symptoms including wrinkles, brittle bones, dull or thin hair, abnormal blood pressure, joint pain, aching muscles, cellulite, loss of mobility, dental issues, facial hallowing, leaky gut and even depression.

### A. Skin Conditions or Diseases

In one aspect, this disclosure provides a method of preventing, ameliorating, or treating a skin condition (e.g., skin aging, senescence, skin pigmentation or skin disease such as acne) in a subject in need thereof. In some embodiments, the method may include identifying a subject having or suspected of having a skin condition (e.g., skin aging, senescence, skin pigmentation or skin disease such as acne). In some embodiments, the skin condition is selected from skin aging, alopecia, scar, acne, actinic damage, dandruff, eczema, fine lines, psoriasis, warts, and wrinkles.

Skin aging occurs via either intrinsic or extrinsic means. The intrinsic source of skin aging is the skin changes associated with chronological age. Extrinsic sources of aging are all of the environmental insults that impact the skin over time, including UV irradiation (photoaging), smoking, air pollution, and other environmental factors. Both intrinsic and extrinsic aging involves multiple biological or pathological pathways, including but not limited to a reduction of the skin's anti-oxidation or free radical scavenging capabilities, and down-regulation of skin's extracellular matrix (ECM) system. This system includes regulation of the production of collagen, elastin, hyaluronic acid, and other molecules that gives the skin the desired appearance and moisture feel.

The defense mechanisms against oxidative stress range from enzymes like superoxide dismutase, catalases, peroxiredoxins, and GSH peroxidases. Also, in the context of intrinsic as well as extrinsic aging, the disruption of the extracellular matrix plays an important role. Enzymes in the extracellular matrix (ECM) are responsible for the processing of elastic fibers, collagens, and proteoglycans.

During aging, the collagen fibrils, elastic fibers, glycoproteins, and glycosaminoglycans are no longer interwoven to form a functional network but form an unorganized dermal-spread agglomeration. This disruption is further aggravated by elastases produced after inflammation or UV exposure and by the activation of matrix metalloproteases (MMPs). Especially MMP1, 2, 3, and 9 are heavily involved in the degradation of the dermal extracellular matrix. During the aging process, these MMPs are upregulated while their inhibitors, namely TIMP1 and 3, are downregulated.

During photoaging, this degradation is significantly accelerated by a process called ECM turnover. UV-irradiation, especially UVA and UVB, results in the production of reactive oxygen species ROS as well as the activation of cell surface receptors leading to an activation of the expression of the transcription factor activation mechanism resulting in the expression of MMP1, 3, and 9 in fibroblasts and keratinocytes. The transcription activation factor protein 1 (AP-1) also inhibits TGF-β, which is responsible for collagen production. The AP-1 mediated MMP expression leads to increased degradation of the ECM. This process is reinforced by the production of ROS.

Multiple skin pigmentation disorders would potentially benefit from miRNA-based therapeutic applications, including vitiligo, albinism, aging spots (such as solar lentigo), freckles, and melasma. Many of these skin disorders involve dysregulation at one or more steps in the melanin synthesis pathway. This multi-step pathway sees the amino acid tyrosine enzymatically converted into dihydroxyphenylalanine, then dopaquinone by tyrosinase (TYR), which is followed by oxidation of dopaquinone into dopachrome. Dihydroxyindole or dihydroxyindole-2-carboxylic acid are formed from dopachrome and finally converted to eumelanin. TYR and its related proteins (such as TYR-related protein 1[TRP1]) are further regulated by the microphthalmia-associated transcription factor (MITF). Multiple miRNAs have been implicated in this signaling pathway.

The formation of structures known as advanced glycation end products (AGE) is another problematic process that can be significantly accelerated by oxidative stress. AGEs originate from the non-enzymatic glycation reaction between sugars and proteins, nucleic acids or lipids.

AGEs are a very heterogeneous group of molecules and can either be ingested through food consumption or formed inside the cell. The cell has specific receptors for AGEs (RAGE). It was shown that RAGEs are highly expressed on mRNA as well as on protein level in fibroblasts and keratinocytes and that expression was increased in sun-exposed skin. AGEs are also heavily connected to oxidative stress. RAGE signaling can directly induce oxidative stress by decreasing the activity of superoxide dismutase (SOD) or indirectly by reducing cellular antioxidant defenses. Via induction of fibroblast activation, the crosslinking of collagen and the increase in metalloproteinase production (MMP 1, 2, and 9), AGEs severely affect the dermis. Concerning the epidermis, it was proposed that AGEs impair the migratory and proliferation abilities of keratinocytes in vitro. There are enzymes in place to counter the genesis of AGEs. One such enzyme is glyoxalase I, which removes α-dicarbonyl compounds, another starting point for AGEs. The decreased activity of this enzyme has been reported during aging. All of these facts paint a very complex picture of the origin and impact of advanced glycation end products.

Keratinocytes and melanocytes, as well as fibroblasts, can become senescent. Senescence-associated beta-galactosidase, a marker of senescence, is found increasingly in aged tissues and aged skin. In the skin, UV-radiation massively induces premature senescence and could, in this way, contribute to skin aging and photoaging. In the dermis, senescent fibroblast activates matrix metalloproteinases and expresses fewer matrix metalloproteinase inhibitors and extracellular matrix components like collagen. Finally, senescent skin cells die by a mechanism that is either described as apoptosis or autophagic programmed cell death. Senescence is initiated after such dramatic incidences as oxidative stress. At high concentrations, ROS are involved in the induction of growth arrest of skin cells.

Skin acne afflicts more than 600 million people globally and frequently ranks as the eighth most common disease among humans. Acne (or acne vulgaris) presents symptoms over a long duration that include greasy skin, pimples, whiteheads, blackheads, and occasional scarring of the skin. These symptoms present when skin oil and dead skin material clog hair follicles. Scientists estimate that more than 80% of all cases of acne vulgaris have a genetic route cause, often implicating tumor necrosis factor (TNF)-a and IL-1-a. Another potential causative factor is the presence of an anaerobic bacterial species on the skin called Propionibacterium acnes, though its specific role in acne development has not been fully elucidated.

b. Methods of Preventing or Treating Skin Conditions or Diseases

The composition and method disclosed herein are useful for regulating and/or improving skin condition. Such regulation of epidermal tissue conditions can include prophylactic and therapeutic regulation. For example, such regulating methods may be directed to thickening dermal tissue and preventing and/or retarding atrophy of skin, preventing and/or retarding the appearance of spider vessels and/or red blotchiness on skin, preventing and/or retarding the appearance of dark circles under the eye, preventing and/or retarding sallowness of skin, preventing and/or retarding sagging of skin, softening and/or smoothing lips, preventing and/or relieving itch of skin, regulating skin texture (e.g. wrinkles and fine lines), and improving skin color (e.g. redness, freckles).

In some embodiments, the method may include administering to the subject an effective amount of an agent capable of reducing a level or activity of at least one of the miR-29a, the miR-29b, and the miR-29c. In some embodiments, the agent may include an antagonist of at least one of miR-29a, miR-29b, and miR-29c. In some embodiments, the antagonist is capable of increasing collagen production in a skin cell by reducing a level or activity of at least one of the miR-29a, the miR-29b, and the miR-29c.

In some embodiments, the composition may include an antagonist of at least one of miR-29a, miR-29b, and miR-29c. In some embodiments, the antagonist is capable of increasing collagen production in a skin cell by reducing a level or activity of at least one of the miR-29a, the miR-29b, and the miR-29c.

In some embodiments, the composition may include: a liposome formulation comprising a phospholipid, a cationic lipid, a pH-dependent cationic lipid, or a combination thereof. In some embodiments, the composition may include a niosome formulation comprising a hydrated nonionic surfactant. In some embodiments, the composition may include a polymer formulation comprising a positively charged polymer.

In some embodiments, the antagonist may include an antagomir of the miR-29a, the miR-29b, or the miR-29c, an antisense oligonucleotide targeting a mature sequence of the miR-29a, the miR-29b, or the miR-29c, an inhibitory RNA molecule, or a combination thereof.

In some embodiments, the miR-29a may include the polynucleotide sequence of SEQ ID NO: 1. In some embodiments, the miR-29b may include the polynucleotide sequence of SEQ ID NO: 2. In some embodiments, the miR-29c may include the polynucleotide sequence of SEQ ID NO: 3.

In some embodiments, the antagonist may include a polynucleotide sequence of SEQ ID NOs: 4-107.

In some embodiments, the inhibitory RNA molecule may include a siRNA or a shRNA that may include the mature sequence of the miR-29a, the miR-29b, or the miR-29c.

In some embodiments, two or more of the antisense oligonucleotides targeting a mature sequence of the miR-29a, the antisense oligonucleotide targeting a mature sequence of the miR-29b, and the antisense oligonucleotide targeting a mature sequence of the miR-29b are carried on the same nucleic acid molecule.

In some embodiments, the liposome formulation may include phospholipid, cholesterol, PEG or a derivative thereof, or a combination thereof. In some embodiments, the liposome formulation may include phospholipid, cholesterol, and PEG or a derivative thereof.

In some embodiments, the phospholipid has a chain of 16 to 22 carbons.

In some embodiments, the phospholipid may include hydrogenated soy phosphatidylcholine (HSPC), distearoylphosphatidylcholine (DSPC), 1,2-dioleoyl-sn-glycero-3-phosphocholine (DOPC), 1,2-distearoyl-3-sn-glycerophosphoethanolamine (DSPE), or dioleoylphosphatidylethanolamine (DOPE).

In some embodiments, the PEG has a molecular weight of 120 Daltons to 5000 Daltons. In some embodiments, the PEG may include PEG[N-(carbamoyl-methoxypolyethylene glycol XXX)-1, 2-distearoyl-sn-glycero-3-phosphoethanolamine sodium salt].

In some embodiments, the liposome formulation may include 40-90 wt % (e.g., 40 wt %, 45 wt %, 50 wt %, 55 wt %, 60 wt %, 65 wt %, 70 wt %, 75 wt %, 80 wt %, 85 wt %, 90 wt %) of phospholipid, 10-60 wt % (e.g., 10 wt %, 15 wt %, 20 wt %, 25 wt %, 30 wt %, 35 wt %, 40 wt %, 45 wt %, 50 wt %, 55 wt %, 60 wt %) of cholesterol, and 0-7 wt % (e.g., 1 wt %, 2 wt %, 3 wt %, 4 wt %, 5 wt %, 6 wt %, 7 wt %) PEG.

In some embodiments, the liposome formulation, the noisome formulation, or the polymer formulation may include a cell-penetrating peptide.

In some embodiments, the cell-penetrating peptide may include an amino acid sequence of SEQ ID NOs: 108.

In some embodiments, the liposome formulation may include: (i) 0-55 wt % (e.g., 5 wt %, 10 wt %, 15 wt %, 20 wt %, 25 wt %, 30 wt %, 35 wt %, 40 wt %, 45 wt %, 50 wt %, 55 wt %) of cationic lipid; (ii) 40-90 wt % (e.g., 40 wt %, 45 wt %, 50 wt %, 55 wt %, 60 wt %, 65 wt %, 70 wt %, 75 wt %, 80 wt %, 85 wt %, 90 wt %) of the cationic lipid and 10-60 wt % (e.g., 10 wt %, 15 wt %, 20 wt %, 25 wt %, 30 wt %, 35 wt %, 40 wt %, 45 wt %, 50 wt %, 55 wt %, 60 wt %) of cholesterol; or (iii) 0-8 wt % (e.g., 1 wt %, 2 wt %, 3 wt %, 4 wt %, 5 wt %, 6 wt %, 7 wt %, 8 wt %) of PEG.

In some embodiments, the cationic lipid may include N-[1-(2,3-dioleoloxy)propyl]-N,N,N-trimethyl ammonium chloride (DOTAP), dimethyldioctadecylammonium (bromide salt) (DDAB), or a combination thereof.

In some embodiments, the liposome formulation may include 0-55 wt % (e.g., 5 wt %, 10 wt %, 15 wt %, 20 wt %, 25 wt %, 30 wt %, 35 wt %, 40 wt %, 45 wt %, 50 wt %, 55 wt %) of pH-dependent cationic lipid. In some embodiments, the pH-dependent cationic lipid may include 1,2-dioleoyl-3-dimethylammonium-propane (DODAP), N-palmitoyl homocysteine (PHC), or a combination thereof.

In some embodiments, the liposome formulation may include an edge activator or inorganic particle. In some embodiments, the edge activator and inorganic particle may include sodium cholate, Span, Tween, and carbonate apatite.

In some embodiments, the liposome formulation may include a skin penetration enhancer.

In some embodiments, the liposome formulation may include 20-45 wt % (e.g., 20 wt %, 25 wt %, 30 wt %, 35 wt %, 40 wt %, 45 wt %) of the skin penetration enhancer. In some embodiments, the skin penetration enhancer may include ethanol.

In some embodiments, the nonionic surfactant may include Span, Tween, brijs, alkyl amides, sorbitan ester, crown ester, or polyoxyethylene alkyl ether.

In some embodiments, the positively charged polymer may include: (a) diethylaminoethylen (DEAE)—Dextran (DEAE-Dextran); (b) linear and branched polyethylenimine (PEI) or derivative thereof; (c) poly(dl-lactide-co-glycolide) (PLGA); (d) Chitosan and modified Chitoson; (e) β-Cyclodextrin; (f) polypeptides; (g) poly{N-[N-(2-aminoethyl)-2-aminoethyl]aspartamide}[PAsp(DET)]; (h) polylysine partially substituted with histidyl residues; and/or (i) linear cationic amphipathic histidine-rich peptide or derivative thereof; and/or a dendrimer.

In some embodiments, the linear cationic amphipathic histidine-rich peptide may include the amino acid sequence of SEQ ID NO: 109 or 110.

In some embodiments, the dendrimer may include poly(amidoamine) (PAMAM), poly(propylenimine) (PPI), or a derivative thereof.

In some embodiments, the composition further may include a positively charged polycation.

In some embodiments, the composition further may include a targeting ligand.

In some embodiments, the target ligand may include (a) a fibroblast growth factor or fibronectin; or (b) a synthetic analog of luteinizing hormone-releasing hormone targeting peptide.

In some embodiments, the composition further may include administering to the subject a second agent. In some embodiments, the second agent may include an anti-inflammatory agent or an antibiotic.

In some embodiments, the method further may include administering to the subject a second agent. In some embodiments, the second agent may include an anti-inflammatory agent or an antibiotic. In some embodiments, the second agent is administered to the subject before, after, or concomitantly with application of the composition.

In some embodiments, the method may include identifying a subject having senescence, skin aging, skin pigmentation or skin disease like acne; and administering to the subject an antagonist of miR-29 expression or function. In some embodiments, administration of a miR-29 antagonist results in the improvement of one or more symptoms of senescence, skin aging, skin pigmentation or skin disease like acne in the subject, or in the delay in the senescence, skin aging, skin pigmentation or skin disease like acne. The one or more improved symptoms may be increased collagen generation, improved skin condition, less scarring, increased quality of life, and decreased disease-related symptom.

Treatment regimens would vary depending on the clinical situation. However, long-term maintenance is suitable in most circumstances.

Ehlers-Danlos syndrome (EDS) is a group of rare genetic disorders affecting humans and domestic animals caused by a defect in collagen synthesis. Depending on the individual mutation, the severity of the disease can vary from mild to life-threatening. Mutations in the ADAMTS2, COL1A1, COL1A2, COL3A1, COL5A1, COL5A2, PLOD1, and TNXB genes cause EDS. Mutations in these genes usually alter the structure, production, or processing of collagen or proteins that interact with collagen. A defect in collagen can weaken connective tissue in the skin, bones, blood vessels, and organs, resulting in the features of the disorder. Thus, collagen deposition induced by miR-29a-c antagonists of the invention would act to replenish the level of normal collagen in EDS patients and alleviate symptoms of the disease. Similarly, administration of an antagonist of miR-29a-c would benefit subjects suffering from vitamin C deficiency or scurvy. Vitamin C deficiency is a disease that results from insufficient intake of vitamin C, which is required for normal collagen synthesis in humans.

Collagen deposition in tissues resulting from the administration of an antagonist of miR-29a-c is beneficial in various cosmetic applications. Effects of aging of the skin produced by natural aging processes or photodamage resulting from over-exposure to the sun could be reduced by administering to a subject in need thereof a miR-29a-c antagonist. Administration of miR-29a-c antagonists may also facilitate the disappearance of stretch marks. Stretch marks are a form of scarring on the skin that is caused by tearing of the dermis. Stretch marks are the result of the rapid stretching of the skin associated with rapid growth (common in puberty) or weight gain (e.g., pregnancy).

In some embodiments, the topical formulation may be applied to skin with cosmetics or applied to skin directly. The tissue to which the disclosed methods may be applied includes facial tissue. Such as forehead tissue, lip, cheek, chin, eyebrow, eyelid, under the eye, or near the mouth, hand tissue, neck tissue, arm tissue, leg tissue, stomach tissue or breast tissue. In some embodiments, the tissue may comprise a wound, a skin graft, scar tissue, wrinkles, lax skin, sun damage, chemical damage, heat damage, cold damage, and/or stretch marks.

In some embodiments, the contacting of the tissue with the miR-29a-c antagonist comprises injection into the tissue, injection into vasculature that feeds the tissue, or topical application. The topical application may be an ointment, cream, gel, salve, or balm. In another embodiment, the method further comprises use of a pressure bandage or dressing. The antagonist of miR-29a-c may be contacted with said tissue more than once. In some embodiments, the antagonist is contacted with the tissue 2, 3, 4, 5, 6, 7, 8, 9, 10, 15, 20, 25, 30, 35, 40, 45, 50, 60, 70, 80, 90 or 100 times. In other embodiments, the antagonist is contacted with the tissue over 2, 3, 4, 5, or 6 days, 1, 2, 3, or 4 weeks, 1, 2, 3, 4, 5, 6, 7, 8, 9, 10 or 11 months, or 1, 2, 3, 3, 4, 5, 6, 7, 8, 9, 10, 15, 20, or 25 years.

In some embodiments, the method further comprises contacting the tissue with a second agent. The second agent may include, but is not limited to, topical vitamin A, topical vitamin C, or vitamin E. In another embodiment, the method further comprises subjecting the tissue to a second treatment. The second treatment may comprise a chemical peel, laser treatment, dermaplaning, or dermabrasion. In another embodiment, the tissue is in a subject that suffers from Ehler's-Danlos syndrome or vitamin C deficiency.

In some embodiments, the method may include the use of miR-29a-c antagonists as profibrotic agents to convert soft plaques in the vasculature into fibrotic tissue to prevent myocardial infarction. Soft plaques are a build-up of lipids containing predominantly cholesterol that lie underneath the endothelial lining of the arterial wall. Recently, it was recognized that these soft plaques are prone to rupture, resulting in the formation of a blood clot, which can potentially block blood flow through the artery and cause a heart attack (i.e., myocardial infarction). It is these soft plaques that are often responsible for causing a healthy subject with no symptoms to Suffer a seemingly unexpected heart attack. After a soft plaque ruptures, the vessel wall heals, and the soft plaque becomes a hard plaque, which rarely causes further problems. Thus, strategies for converting soft plaques into fibrotic tissue would prevent the soft plaques from rupturing and possibly inducing a myocardial infarction.

As described in detail above, inhibition of miR-29a-c leads to an increase in collagen deposition and the formation of fibrotic tissue. Accordingly, this disclosure also provides a method for increasing fibrotic tissue formation in the wall of a vessel comprising delivering an antagonist of miR-29a-c to one or more soft plaque sites in the vessel wall, wherein the soft plaque is converted to fibrotic tissue following delivery of the antagonist of miR-29a-c. Soft plaques can be identified by methods known in the art, including, but not limited to, intravascular ultrasound and computed tomography (Sahara et al. (2004) European Heart Journal, Vol. 25: 2026-2033; Budhoff (2006) J. Am. Coll. Cardiol. Vol. 48: 319-321; Hausleiter et al. (2006) J. Am. Coll. Cardiol. Vol. 48: 312-318). Any of the miR-29a-c antagonists described herein are suitable for use in the method.

The miR-29a-c antagonist may be delivered to one or more soft plaque sites by direct injection or by using a catheter or a device that isolates the coronary circulation. In one embodiment, the miR-29a-c antagonist is delivered to one or more soft plaque sites by a medical device used in vascular surgery, such as a stent or balloon. The miR-29 antagonist may be coated on a metal stent to form a drug-eluting stent. A drug-eluting stent is a scaffold that holds open narrowed or diseased arteries and releases a compound to prevent cellular proliferation and/or inflammation. miR-29a-c antagonists may be applied to a metal stent embedded in a thin polymer for release of the miR-29a-cover time. Methods of coating stents with therapeutic compounds are known in the art. See, e.g., U.S. Pat. Nos. 7,144,422; 7,055,237; and WO 2004/004602, which are here incorporated by reference in their entireties. In some embodiments, the miR-29a-c may be used in combination with other anti-restenosis compounds to produce a formulation for incorporation into drug-eluting stents and balloons. Suitable compounds for use in combination with the antagonists of miR-29a-c include, but are not limited to, paclitaxel, rapamycin (sirolimus), tacrolimus, Zotarolimus, everolimus, docetaxel, pimecrolimus, and derivatives thereof.

c. Methods of Preventing or Treating Other Conditions or Diseases Associated with Collagen Deficiency

The agents, formulations, products and methods disclosed herein can be used for improving conditions associated with collagen deficiency or for treating diseases or disorders associated with collagen deficiency in addition to skin conditions or disorders in a subject in need thereof. The subject may be one suffering from or be predisposed to a condition characterized by, or associated with a collagen deficiency or collagen malfunction in nails, hairs, joint, bone, or other connective tissues.

One aspect of this disclosure involves enhancing mechanical properties of tissue (such as nails, bones, tendons, ligaments, and cartilage), improving tissue mechanical properties, and treating related musculoskeletal conditions or injuries. The methods and compositions described herein may find use as in treating diseases and syndromes related to collagen or elastin crosslinking deficiency (e.g., osteolathyrism, Ehlers Danlos Syndrome, etc.).

In one examples, the agents, formulations, products and methods disclosed are useful in the fields of orthopedic surgery, rheumatology, sport and rehabilitation medicine. The methods can permit formation of de novo and regeneration of diseased or injured bone or cartilage, particularly articular cartilage. The methods also permit in vivo or ex vivo regeneration of bone, cartilage or usable cartilage grafts removed from the diseased or injured joint, regenerating such bone, cartilage or graft to a degree where both the mechanical and biochemical properties of the bone or cartilage are restored to normal levels and replacing the graft into the joint once cartilage collagen matrix is restored. Additionally, the method permits the in vitro treatment of cartilage and bone cells and cell cultures into functional tissue suitable for transplantation and de novo formation and production of healthy normally functioning cartilage and other mesenchymally-derived cells.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, a “subject” refers to a human and anon-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental animal or animal suitable as a disease model.

“Treating” or “treatment” as used herein refers to administration of a compound or agent to a subject who has a disorder with the purpose to cure, alleviate, relieve, remedy, delay the onset of, prevent, or ameliorate the disorder, the symptom of a disorder, the disease state secondary to the disorder, or the predisposition toward the disorder.

An “effective amount” or “therapeutically effective amount” refers to an amount of the compound or agent that is capable of producing a medically desirable result in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route.

The term “in vitro” refers to events that occur in an artificial environment, e.g., in a test tube or reaction vessel, in cell culture, etc., rather than within a multi-cellular organism. As used herein, the term “in vivo” refers to events that occur within a multi-cellular organism such as a non-human animal.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “decrease,” “reduced,” “reduction,” “decrease,” and “inhibit” are all used herein generally to mean a decrease by a statistically significant amount. However, for avoidance of doubt, these terms refer to a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “modulate” is meant to refer to any change in biological state, i.e. increasing, decreasing, and the like.

The terms “increased”, “increase” “enhance” and “activate” are all used herein to generally mean an increase by a statically significant amount; for the avoidance of any doubt, these terms refer to an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit]“per kg (or g, mg etc.) bodyweight”, even if the term “bodyweight” is not explicitly mentioned.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

The terms “inhibit” and “antagonize,” as used herein, mean to reduce a molecule, a reaction, an interaction, a gene, an mRNA, and/or a protein's expression, stability, function or activity by a measurable amount or to prevent entirely. Inhibitors are compounds that, e.g. bind to, partially or totally block stimulation, decrease, prevent, delay activation, inactivate, desensitize, or down-regulate a protein, a gene, and an mRNA stability, expression, function, and activity, e.g., antagonists.

“Parenteral” administration of a composition includes, e.g., subcutaneous (s.c.), intravenous (i.v.), intramuscular (i.m.), or intrastemal injection, or infusion techniques.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art, including, but not limited to, intravenous, oral, aerosol, parenteral, ophthalmic, pulmonary, and topical administration.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of this disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

The term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of this disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated or otherwise clearly contradicted by context. In regard to any of the methods, the steps of the method may occur simultaneously or sequentially. When the steps of the method occur sequentially, the steps may occur in any order, unless noted otherwise. In cases where a method comprises a combination of steps, each and every combination or sub-combination of the steps is encompassed within the scope of the disclosure, unless otherwise noted.

Each publication, patent application, patent, and other reference cited herein is incorporated by reference in its entirety to the extent that it is not inconsistent with the present disclosure. Publications disclosed herein are provided solely for their disclosure prior to the filing date of this disclosure. Nothing herein is to be construed as an admission that this disclosure is not entitled to antedate such publication by virtue of prior invention. Further, the dates of publication provided may be different from the actual publication dates, which may need to be independently confirmed.

It is understood that the examples and embodiments described herein are for illustrative is purposes only and that various modifications or changes in light thereof will be suggested to persons skilled in the art and are to be included within the spirit and purview of this application and scope of the appended claims.

### EXAMPLES

### Example 1 Collagen I Expression is Downregulated by miR-29 and Upregulated by Antagonists of miR-29

To better understand the role of miR-29 family in collagen I regulation, an immunofluorescence assay was performed to estimate the effects of miR-29 on collagen I expression in human dermal fibroblasts. Primary human dermal fibroblasts culture was seeded in wells and transfected for immunofluorescence imaging. The results show that miR-29 (Sample 1) inhibited collagen I expression at three concentrations (20, 30, and 50 pM), while miR-29 antagonists (Sample 2) promoted collagen I synthesis at 30 and 40 pM. Thus, miR-29 can be considered as a negative regulator of collagen I synthesis, and inhibiting miR-29 promotes collagen I synthesis.

### Example 2 Collagen III Expression is Downregulated by miR-29 and Upregulated by Antagonists of miR-29

Like collagen I, an immunofluorescence assay was performed to estimate the effects of miR-29 on collagen III expression in human dermal fibroblasts. Primary human dermal fibroblasts culture was seeded in wells and transfected for immunofluorescence imaging. The results show that miR-29 (Sample 1) inhibited collagen III expression at three concentrations (30, 40, and 50 pM), while miR-29 antagonists (Sample 2) promoted collagen III synthesis at 20, 30, and 40 pM. Thus, miR-29 can be considered as a negative regulator of collagen III synthesis, and inhibiting miR-29 promotes Collagen III synthesis.

### Example 3 Quantitative Analysis of miR-29 Regulation of Collagen I Synthesis

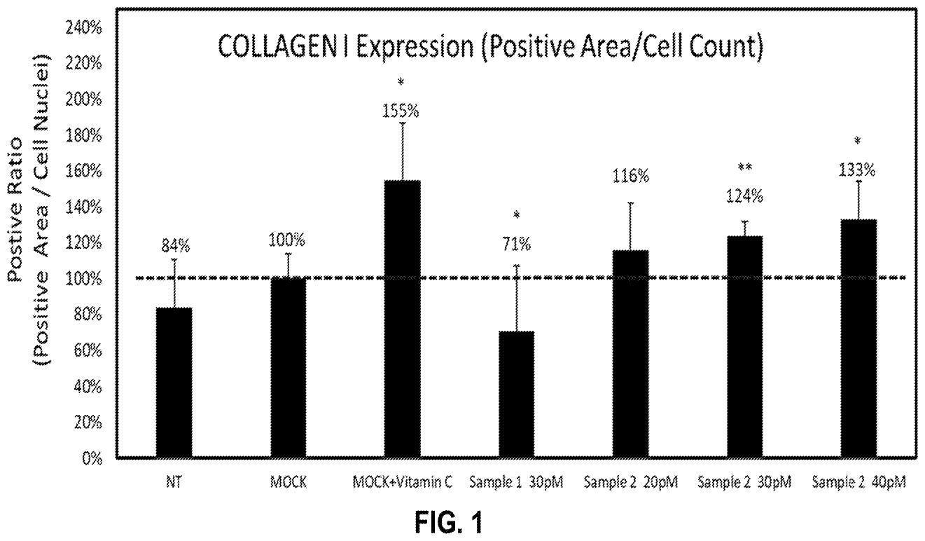

For further quantification of collagen I synthesis after miR-29 upregulation or downregulation, collagen expression positive ratio was calculated based on immunofluorescence imaging (see FIG. 1). miR-29 was applied at 30 pM, and antagonists of miR-29 were applied at 20, 30, and 40 pM. The results show significant inhibition of collagen I synthesis in dermal fibroblasts transfected with 30 pM of miR-29 (P<0.05 vs. Mock). Also, introducing antagonists of miR-29 improved collagen synthesis at all three concentrations, and higher concentrations of miR-29 antagonists further improved collagen I synthesis (P<0.05 vs. Mock at both 30 and 40 pM). Although the promotion of collagen I synthesis by miR-29 antagonists is not as good as vitamin C in selected concentrations, the results of the experiment show that miR-29 antagonists increased collagen I synthesis. Thus, miR-29 antagonists have an application in treating collagen dysfunction-related symptoms, such as senescence, skin aging, skin pigmentation, and skin disease such as acne.

### Example 4 Delivery System 1: A Cationic Liposome-Based Delivery System

To synthesize the liposome, all components (i.e., DSPC, DSPE-PEG2000-amine, and DOTAP) were mixed in ethanol at a molar ratio such as 7:1:2, 8:1:1, 6:1:3, or 7:1.5:1.5. Then the organic phase containing the liposome components was mixed with the water phase containing a miR-29 antagonist using a microfluidic device. The organic components formed a liposome structure with the miR-29 antagonist loaded therein. The liposomes encapsulating the miR-29 antagonist were then run through an ultrafiltration device for washing. After ultrafiltration, the liposomes were collected, and their particle sizes were measured. The liposomes have a size from about 100 to about 300 nm (e.g., about 280 nm).

To test cellular uptake of the liposomes encapsulating the miR-29 antagonist, human dermal fibroblasts (5×10³ cells per well) were seeded in an 8-well cell culture chamber slide and incubated overnight. Cells were exposed to assembled nanocarriers at 20, 30, and 40 pM, and incubated for 6, 12, or 24 h. One control group was also treated with a free miR-29 antagonist at the same antagonist concentration as the experimental groups. Another control group was treated with an empty carrier at the same carrier concentration as the experimental groups. Then, the cells were washed with DPBS and fixed. All samples were then tested for collagen (i.e., collagen I and III) expression using immunofluorescence.

To test skin penetration of the liposomes encapsulating the miR-29 antagonist, human skin (pig skin can also be used) prepared for research was cut into a specific size and placed on a 6-well plate containing 2 mL of DPBS, which was used to prevent the skin from drying out during the experiment, a miR-29 antagonist carried by liposomes, naked miR-29 antagonist, and DPBS (control) loaded with RNA at the same concentration as the miR-29 antagonist were sprinkled on the skin and incubated at 37° C. in a humidified 5% CO₂ incubator. The surface of the skin was washed two to three times with DPBS, and the skin tissues were frozen for cryosectioning. Tissue sections cut to 10 μm thickness were prepared, and optical and fluorescence analysis was performed using a fluorescent microscope to examine skin penetration and collagen (i.e., collagen I and III) expression.

Next, in vivo activity of the liposomes encapsulating the miR-29 antagonist was tested in an animal model. 10-week-old female mice were randomized into four different groups (12 mice in each group). Then 40 pM of miR-29 antagonist carried by liposomes, naked miR-29 antagonist, vitamin C or PBS were respectively applied to the dorsal skin of the mice every is day. 7, 14, and 21 days after treatment, the mice were anesthetized with isoflurane. The dermal fibroblasts sample were isolated to measure the collagen expression. All animal procedures were diligently designed and performed in conformity with institutional guidelines and protocols for the care and use of laboratory animals.

### Example 5 Delivery System: Skin-Penetration Peptide+Polymer

The formulation combines the advantages of SCP and PAE for effective transdermal gene delivery. The SCP-PAE micelle is a conjunction of cell-penetrating peptide (CCP) and synthesized poly (β-amino ester) (PAE). The PAE part consists of 3-Amino-1-pentanol, 1,4-butane diacrylate, chloroform, hyaluronic acid (MW=8000), N-hydroxysuccinimide, and 1-ethyl-3-(3-dimethylaminopropyl) carbodiimide hydrochloride. To synthesize micelles, 3-aminopropanol and 1,4-butane diacrylate (at a molar ratio, e.g., 3.73:10 or 4:10) were stirred at 55-65° C. (e.g., 60° C.), then heated to reflux for homogenization for 1.5-2 h (e.g., 1.5 h). After the completion of the reaction, the product was dissolved in ethanol (e.g., 20 mL). Decuple pre-cooled diethyl ether solution was added to precipitate the product. The precipitated product was repeatedly filtered three times, and the filtered product was vacuum dried for more than 24 h to obtain a higher purity polymer polyurethane. SCP (ACTGSTQHQCG (SEQ ID NO: 111) or other possible formulation) was synthesized. To assemble PAE and SCP, polymer polyurethane and SCP were dissolved in ultrapure water at a molar ratio of 1:2 (other ratios were also tested), and stirred for 6 h. The product was subsequently subject to dialysis for 24 h. Lyoprotectant was then added and freeze-dried for 24 h to obtain the product.

To load the miR-29 antagonist in the micelles, the micelles (1 mg) was dissolved in water (1 mL), and then miR-29 antagonist (20 μg) was put into micelles solution (10 μL). 30 μL of the resulting solution was incubated at 37° C. for 15 min. For the characterization, the particle size and zeta potential of the micelle were measured.

To test cellular uptake of the miR-29 antagonist loaded micelles, human dermal fibroblast cells were seeded on 24-well plate containing 10% PBS. After cells were incubated overnight, 40 pM PBS, naked miR-29 antagonist, and miR-29 antagonist loaded micelles (final miR-29 antagonist concentration: 50 pM/mL) were added to the media, respectively. Cells were incubated for 3, 6, and 9 h, respectively. The media was subsequently discarded, and the cells were washed with cold PBS three times. The cells were then collected for immunofluorescence analysis to measure the collagen expression.

To test skin penetration of the miR-29 antagonist loaded micelles, the skins of nude mice (10 weeks old) were used for the in vitro penetration test of SCP-PAE-miR-29 antagonist. Mice were anesthetized using 10% chloralhydrate, and then the dorsal skin hair of all mice was removed using hair removal cream. The skin was used once it was removed from mice. Skin was cut to a suitable size and put into the diffusion cells containing PBS (pH 7.4). Skin samples were maintained at 37° C. The volume of SCP-PAE-miR-29 antagonist loaded micelles solution was 1 mL (final miR-29 antagonist concentration: 50 pM/mL). The supply compartment was sealed to avoid the spread of liquid. Diffuse compartment was stirred constantly in a water bath at 37° C. (300 r/min). After 24 h, the diffusion cell device was removed, and the skin was gently wiped using a cotton swab and then immediately placed on a glass slide to observe the distribution of the collagen expression in the skin.

### Example 6 Delivery System: Additional Example of the Liposome-Based Delivery System

This liposome carrier is based on a liposome called DDC642, capable of delivering RNAi molecules to the epidermis of impaired and intact human skin, without targeting the dermis or circulatory system. The formulation includes DOTAP (2,3-dioleoyloxy-propyl-trimethylammonium chloride), DOPE (1,2-dioleoyl-snglycero-3-phosphoethanolamine), and Tween 20/80 (TW20/80).

To synthesize the liposome carrier, lipids were dissolved in chloroform, and TW20/80 was dissolved in sterile distilled water (DOTAP:DOPE:Tween=6:4:2 or 6:5:1). The organic phase and water phase were mixed using a microfluidic device. After mixing the components, the solvent was removed by rotary vacuum evaporation above a lipid transition temperature. The resulting film was hydrated with 30% EtOH. After overnight incubation, vesicles were extruded through a 100 nm polycarbonate membrane filter. Corresponding lipoplexes (LPX) were prepared by diluting the RNAi molecules with a HEPES buffer. Liposomes were added under vortex mixing. Average particle size and zeta potential were determined.

To test cellular uptake of the miR-29 antagonist loaded liposomes, human dermal fibroblast cells were housed in a keratinocyte growth medium in an incubator at 37° C., 99% humidity, and 10% CO₂. After 24 h growth, cells were seeded at approximately 3×10⁵ cells/well in 6-well flatbottom cell culture plates. Prior to adding carrier and miR-29 antagonist, fibroblast cells were washed with PBS, then LPX was added to the cells. After 24 h (anti-miR) incubation in minimal medium, complexes were removed, and the medium was replaced. The final concentration of miR-29 antagonist was determined as about 50 nM for fibroblast uptake. Cellular uptake efficiencies were determined after 48 hours by immunofluorescence and Q-PCR. For Q-PCR, total RNA was extracted using RNA extraction kit according to the manufacturer's requirements. DNase treatment was performed, and first-strand cDNA was generated by reverse transcription using cDNA synthesis kit. miR-29 antagonist expression levels were determined using a SYBR Green I reverse transcription PCR assay with primers designed for miR-29 antagonist sequence. PCR reactions were performed on a Q-PCR device using SYBR Green I master mix. Expression levels were normalized using the geometric mean of one (or more) reference gene.

To test skin penetration of the miR-29 antagonist-loaded liposomes, human excised skin was cleaned with PBS and used immediately after being obtained. LPXs containing liposome-carried miR-29 antagonist were applied onto full thickness skin under non-occlusive conditions for 6 h at 4° C. Untreated skin was used as negative control. Then 7 μm cross sections of skin were made and analyzed after embedding in optimal cutting temperature compound. Immunofluorescence imaging on collagen expression was obtained by microscopy. Processing was applied equally to all images using Image J software. The experiment was performed in triplicate (or duplicate). Therapeutic efficacy was analyzed with collagen expression. Skin samples were handled according to manufacturer's guidelines.

### Example 7 Delivery System: A pH-Responsive Delivery System

This delivery system is based on a dual-function polyamine lipid derivative, dioleylphosphate-diethylenetriamine (DOP-DETA) conjugate. DOP-DETA has positively charged diethylenetriamine residue that contributes not only to the capture of siRNA, but also to the interaction between the lipid and the cell membrane, as well as to the interaction between the lipid and the endosomal membrane. Furthermore, the unsaturated carbon chain in DOP-DETA contributes to higher membrane fluidity and induces membrane fusion.

DOP-DETA was synthesized as follows. A mixture of oleyl alcohol (e.g., 200 L, 0.63 mmol) and diphenyl phosphite (e.g., 61 L, 0.31 mmol) was heated at 120° C. for 1-2 h (e.g., 1.5 h) under reduced pressure. The resultant mixture was subjected to silica gel chromatography with a hexane/diethyl ether (1/1) eluent, giving dioleylphosphite. Then, to a solution of anhydrous dichloromethane dissolving diethylenetriamine (e.g., 107 L, 1 mmol) and N,N-diisopropylethylamine (e.g., 35 L at 0.2 mmol) was added dioleylphosphite (58 mg, 0.1 mmol) with stirring at 0° C. for 2-4 h (e.g., 3 h). After solvent removal, the resultant was dried under reduced pressure. The resultant solid was washed with distilled water. The solid residue was dissolved in chloroform and subject to column chromatography by use of amino-modified silica gel with a chloroform/methanol (39/1) eluent. DOP-DETA was obtained as a white solid.

To prepare the delivery system, DOP-DETA, DPPC, and cholesterol were dissolved in tertbutyl alcohol and lyophilized. Then, the liposomes and RNA were mixed gently and incubated for 20 min at room temperature to obtain the final product. Particle size, polydispersity index (PDI), and zeta-potential of the assembled delivery system was measured.

To measure surface charge at various pH values of the miR-29 antagonist-loaded DOP-DETA, diluted DOP-EDTA-RNA (0.15 mM DL/RNA) was prepared (with RNase-free water), and the ξ-potential at pH 4 to pH 9 was measured with a Multi-Purpose Titration device (Malvern) and a Zetasizer Nano ZS analyzer, whole measurements were carried out following the manufacturer's instructions.

To test cellular uptake of the miR-29 antagonist-loaded DOP-DETA, human dermal fibroblast cells were seeded on a 24-well plate containing 10% PBS in an incubator at 37° C., 99% humidity, and 10% CO₂. After cells were incubated overnight, then 40 pM PBS, naked miR-29 antagonist, and miR-29 antagonist loaded DOP-DETA (final miR-29 antagonist concentration: 50 pM/mL) were added to the media, respectively. Cells were incubated for 3, 6, and 9 h, respectively. Cellular uptake efficiencies were determined after 48 hours by immunofluorescence, as mentioned in the above samples.

To test skin penetration of the miR-29 antagonist-loaded DOP-DETA, human excised skin was cut into a specific size and placed on a 6-well plate containing 2 mL of PBS, which was used to prevent the skin from drying out during the experiment. miR-29 antagonist-loaded DOP-DETA (RNA concentration 50 pM/cm²), naked miR-29 antagonist (RNA concentration 50 pM/cm²), and PBS (control) loaded with same RNA concentration were sprinkled on the skin. Skin samples were maintained at 37° C. Immunofluorescence imaging was performed on skin samples at 3, 6, 9, 12, 24, and 72 hours after sprinkling. The delivery efficiency was determined by the illustrated collagen expression.

The disclosure is not limited in scope by the specific embodiments described herein. Various modifications of the invention in addition to those described herein will become apparent to those skilled in the art from the foregoing description and the accompanying figures. Such modifications are intended to fall within the scope of the appended claims.

1. A composition comprising an agent for treating a condition or disorder associated with collagen deficiency, for preventing or treating a skin disease or disorder, or for improving a skin condition.

2. The composition of claim 1, wherein the agent comprises an antagonist of at least one of miR-29a, miR-29b, and miR-29c, wherein the antagonist is capable of increasing collagen production in a skin cell by reducing a level or activity of at least one of the miR-29a, the miR-29b, and the miR-29c.

3. The composition of claim 1, further comprising a liposome formulation comprising a phospholipid, a cationic lipid, a pH-dependent cationic lipid, or a combination thereof, a niosome formulation comprising a hydrated nonionic surfactant; or a polymer formulation comprising a positively charged polymer.

4. The composition of claim 2, wherein the antagonist comprises an antagomir of the miR-29a, the miR-29b, or the miR-29c, an antisense oligonucleotide targeting a mature sequence of the miR-29a, the miR-29b, or the miR-29c, an inhibitory RNA molecule, or a combination thereof.

5. The composition of claim 2, wherein the miR-29a comprises the polynucleotide sequence of SEQ ID NO: 1.

6. The composition of claim 2, wherein the miR-29b comprises the polynucleotide sequence of SEQ ID NO: 2.

7. The composition of claim 2, wherein the miR-29c comprises the polynucleotide sequence of SEQ ID NO: 3.

8. The composition of claim 2, wherein the antagonist comprises a polynucleotide sequence selected from the group consisting of SEQ ID NOs: 4-107.

9. The composition of claim 4, wherein the inhibitory RNA molecule comprises a siRNA or a shRNA that comprises the mature sequence of the miR-29a, the miR-29b, or the miR-29c.

10. (canceled)

11. The composition of claim 3, wherein the liposome formulation comprises phospholipid, cholesterol, PEG or a derivative thereof, or a combination thereof.

12. (canceled)

13. (canceled)

14. (canceled)

15. (canceled)

16. (canceled)

17. (canceled)

18. The composition of claim 3, wherein the liposome formulation, the noisome formulation, or the polymer formulation comprises a cell-penetrating peptide.

19. (canceled)

20. The composition of claim 3, wherein the liposome formulation comprises: (i) 0-55 wt % of cationic lipid; (ii) 40-90 wt % of the cationic lipid and 10-60 wt % of cholesterol; or (iii) 0-8 wt % of PEG.

21. (canceled)

22. (canceled)

23. (canceled)

24. The composition of claim 3, wherein the liposome formulation comprises an edge activator or inorganic particle.

25. (canceled)

26. The composition of claim 3, wherein the liposome formulation comprises a skin penetration enhancer.

27. (canceled)

28. (canceled)

29. (canceled)

30. (canceled)

31. (canceled)

32. (canceled)

33. The composition of claim 1, wherein the composition further comprises a positively charged polycation.

34. The composition of claim 1, wherein the composition further comprises a targeting ligand.

35. (canceled)

36. The composition of claim 1, further comprising a second agent.

37. (canceled)

38. (canceled)

39. A kit or device comprising the composition of claim 1.

40. (canceled)

41. (canceled)

42. (canceled)

43. A method for treating a condition or disorder associated with collagen deficiency, for preventing, ameliorating, or for treating a skin condition in a subject, comprising administering to the subject a therapeutically effective amount of the composition of claim 1.

44. A method of increasing collagen production in a skin cell of a subject, comprising administering to the subject a therapeutically effective amount of the composition of claim 1.

45. (canceled)

46. (canceled)

47. (canceled)

48. (canceled)

49. (canceled)

50. (canceled)
